(19) United States
(12) Patent Application Publication
(10) Pub. No.: US 2022/0305048 A1
(43) Pub. Date: Sep. 29, 2022

(54) USE OF HEPARIN TO PROMOTE TYPE 1 INTERFERON SIGNALING

(71) Applicant: Dana-Farber Cancer Institute, Inc., Boston, MA (US)

(72) Inventors: Shriram Sundararaman, Charlottesville, VA (US); Erik Knelson, Brookline, MA (US); Shunsuke Kitajima, Allston, MA (US); David Barbie, Andover, MA (US); Saemi Han, Gwancheon-si (KR)

(73) Assignee: Dana-Farber Cancer Institute, Inc., Boston, MA (US)

(21) Appl. No.: 17/638,150

(22) PCT Filed: Aug. 26, 2020
(86) PCT No.: PCT/US2020/047985

§ 371 Date: Feb. 24, 2022

Related U.S. Application Data
(60) Provisional application No. 63/039,383, filed on Jun. 15, 2020.
(60) Provisional application No. 62/891,822, filed on Aug. 26, 2019.

(51) Int. Cl. A61K 31/727; A61K 45/06; A61K 9/00; A61P 35/00

(52) U.S. Cl. CPC A61K 31/727; A61K 45/06; A61K 9/0019; A61P 35/00

(57) ABSTRACT

Disclosed herein are methods for treating a subject having cancer by coadministering a stimulator of interferon signaling and a heparin polysaccharide. Also disclosed herein are pharmaceutical compositions that include a stimulator of interferon signaling and a heparin polysaccharide.

### GOVERNMENT SUPPORT

This invention was made with government support under Contract No. NCI-RO1 CA190394, awarded by the National Cancer Institute (NCI) and under Contract No. NIH-U01 CA214381, awarded by the National Institutes of Health (NIH). The government has certain rights in the invention.

### BACKGROUND OF THE INVENTION

Cancer is the second leading cause of death in the USA and globally. It is a group of a diseases characterized by abnormal cell growth, and in some cases, metastasis. There are various treatment approaches for cancer, one of the most common being chemotherapy—the use of drugs to kill cancerous cells, slow disease progression, combat metastasis, treat symptoms (palliative chemotherapy), etc. Chemotherapy can be systemic or local. One of the major challenges with these treatments is their reliance on differential toxicity for cancerous cells versus normal cells. “Cancer immunotherapy” is a term that refers to therapies that artificially stimulate the immune system to combat cancer. It is a newer subspecialty of oncology with the potential to resolve the clinical, societal, and financial burden of treating cancer.

Heparin is an anticoagulant (or blood thinner) that can be naturally produced by basophils and mast cells. It is typically used to treat or prevent disorders relating to clotting, such as, deep vein thrombosis, pulmonary embolism, and arterial thromboembolism.

### SUMMARY OF THE INVENTION

The innate immune system is an emerging target for tumor immunotherapy. The present disclosure is based, at least in part, on methods of treating a subject having cancer, comprising administering a therapeutically effective amount of a stimulator of interferon signaling, including but not limited to a stimulator of interferon gene (STING) agonist, and a therapeutically effective amount of a heparin polysaccharide.

Accordingly, one aspect of the present disclosure provides a method of treating a subject having cancer, comprising administering to the subject a therapeutically effective amount of a stimulator of interferon signaling and a therapeutically effective amount of a heparin polysaccharide, wherein the heparin polysaccharide has reduced anticoagulant activity. In some embodiments, the heparin polysaccharide is at least one of desulfated and N-acetylated. In some embodiments, the heparin polysaccharide is at least one of N-desulfated and O-desulfated. In some embodiments, the heparin polysaccharide is at least one of 2-O desulfated, 3-O desulfated, and 6-O desulfated. In some embodiments, the heparin polysaccharide comprises a glycol-split monomer. In some embodiments, the heparin polysaccharide lacks a unique pentasaccharide sequence, wherein the unique pentasaccharide sequence has the following general structure:

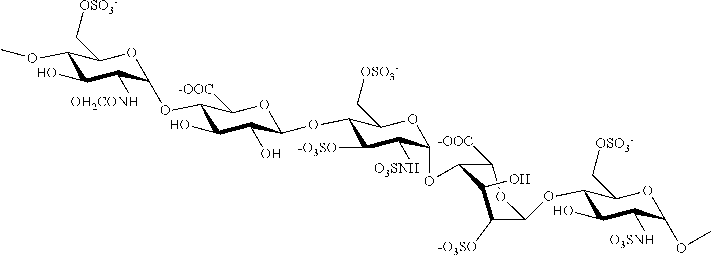

In some embodiments, the heparin polysaccharide is administered locally, intratumorally, or systemically. In some embodiments, the stimulator of interferon signaling is administered locally, intratumorally, or systemically. In some embodiments, the heparin polysaccharide is low molecular weight heparin. In some embodiments, the stimulator of interferon signaling is selected from the group consisting of interferon alpha, interferon beta, STING agonists, TLR agonists, and oncolytic viruses. In some embodiments, when the stimulator of interferon signaling is a STING agonist, it is selected from the group consisting of cyclic GMP-AMP (cGAMP), ganciclovir, ADU-S100, and CMA.

In some embodiments, the method further comprises administering to the subject a chemotherapeutic agent. In some embodiments, the chemotherapeutic agent is a checkpoint inhibitor. In some embodiments, the chemotherapeutic agent is a programmed cell death protein 1 (PD-1) inhibitor or a programmed death-ligand 1 (PD-L1) inhibitor. In some embodiments, the cancer is selected from the group consisting of carcinoma, lymphoma, blastoma, sarcoma, and leukemia. In some embodiments, the cancer is selected from the group consisting of cancers of the lung, bone, pancreas, skin, head, neck, uterus, ovaries, stomach, colon, breast, esophagus, small intestine, bowel, endocrine system, thyroid gland, parathyroid gland, adrenal gland, urethra, prostate, penis, testes, ureter, bladder, kidney or liver; rectal cancer, cancer of the anal region, carcinomas of the fallopian tubes, endometrium, cervix, vagina, vulva, renal pelvis, renal cell, sarcoma of soft tissue, myxoma, rhabdomyoma, fibroma, lipoma, teratoma, cholangiocarcinoma, hepatoblastoma, angiosarcoma, hemangioma, hepatoma, fibrosarcoma, chondrosarcoma, myeloma, chronic or acute leukemia, lymphocytic lymphomas, primary CNS lymphoma, neoplasms of the CNS, spinal axis tumors, squamous cell carcinomas, synovial sarcoma, malignant pleural mesotheliomas, brain stem glioma, pituitary adenoma, meningioma, bronchial adenoma, chondromatous hanlartoma, inesothelioma, Hodgkin's Disease, brain (gliomas), glioblastomas, astrocytomas, glioblastoma multiforme, Bannayan-Zonana syndrome, Cowden disease, Lhermitte-Duclos disease, Wilm's tumor, Ewing's sarcoma, Rhabdomyosarcoma, ependymoma, medulloblastoma, melanoma, ovarian, pancreatic, adenocarcinoma, ductal madenocarcinoma, adenosquamous carcinoma, small cell lung cancer, acinar cell carcinoma, glucagonoma, insulinoma, prostate, sarcoma, osteosarcoma, giant cell tumor of bone, thyroid, lymphoblastic T cell leukemia, chronic myelogenous leukemia, chronic lymphocytic leukemia, hairy-cell leukemia, acute lymphoblastic leukemia, acute myelogenous leukemia, chronic neutrophilic leukemia, acute lymphoblastic T cell leukemia, plasmacytoma, Immunoblastic large cell leukemia, mantle cell leukemia, multiple myeloma, megakaryoblastic leukemia, multiple myeloma, acute megakaryocyte leukemia, pro myelocytic leukemia, erythroleukemia, malignant lymphoma, hodgkins lymphoma, non-hodgkins lymphoma, lymphoblastic T cell lymphoma, Burkitt's lymphoma, follicular lymphoma, neuroblastoma, bladder cancer, urothelial cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor) and testicular cancer.

In some embodiments, the cancer is selected from the group consisting of small cell lung cancer, non-small cell lung cancer, mesothelioma, meningioma, and triple negative breast cancer.

Another aspect of the present disclosure provides a method of treating a subject having cancer, comprising administering to the subject a therapeutically effective amount of a stimulator of interferon signaling and a therapeutically effective amount of a heparin polysaccharide, wherein the subject is not receiving concurrent antithrombotic therapy or thrombolytic therapy. In some embodiments, the heparin polysaccharide is at least one of desulfated and N-acetylated. In some embodiments, the heparin polysaccharide is low molecular weight heparin. In some embodiments, the antithrombotic therapy is an anticoagulant therapy. In some embodiments, the cancer is meningioma, glioma, medulloblastoma, pituitary adenomas, primary central nervous system (CNS) lymphomas, or a cancer associated with central nervous system (CNS) germ cell tumors. In some embodiments, the cancer is small cell lung cancer. In some embodiments, the subject has or is at risk of having intracranial bleeding. In some embodiments, the subject has or is at risk of having hepatic damage or hepatic failure. In some embodiments, the subject is undergoing surgery on the brain or CNS. In some embodiments, the heparin polysaccharide is administered locally, intratumorally, or systemically. In some embodiments, the stimulator of interferon signaling is administered locally, intratumorally, or systemically. In some embodiments, the stimulator of interferon signaling is selected from the group consisting of interferon alpha, interferon beta, STING agonists, TLR agonists, and oncolytic viruses. In some embodiments, when the stimulator of interferon signaling is a STING agonist, it is selected from the group consisting of cyclic GMP-AMP (cGAMP), ganciclovir, ADU-S100, and CMA. In some embodiments, the method further comprises administering to the subject a chemotherapeutic agent. In some embodiments, the chemotherapeutic agent is a checkpoint inhibitor. In some embodiments, the chemotherapeutic agent is a PD-1 inhibitor or a PD-L1 inhibitor.

Another aspect of the present disclosure provides a method of treating a subject having cancer, comprising administering to the subject a therapeutically effective amount of a stimulator of interferon signaling and a therapeutically effective amount of a heparin polysaccharide, wherein the heparin is administered locally to the cancer or intratumorally. In some embodiments, the stimulator of interferon signaling is administered locally to the cancer or intratumorally. In some embodiments, the stimulator of interferon signaling is selected from the group consisting of interferon alpha, interferon beta, STING agonists, TLR agonists, and oncolytic viruses. In some embodiments, when the stimulator of interferon signaling is a STING agonist, it is selected from the group consisting of cyclic GMP-AMP (cGAMP), ganciclovir, ADU-S100, and CMA.

In some embodiments, the method further comprises administering to the subject a chemotherapeutic agent. In some embodiments, the chemotherapeutic agent is a checkpoint inhibitor. In some embodiments, the chemotherapeutic agent is a PD-1 inhibitor or PD-L1 inhibitor. In some embodiments, the cancer is selected from the group consisting of carcinoma, lymphoma, blastoma, sarcoma, and leukemia. In some embodiments, the cancer is selected from the group consisting of cancers of the lung, bone, pancreas, skin, head, neck, uterus, ovaries, stomach, colon, breast, esophagus, small intestine, bowel, endocrine system, thyroid gland, parathyroid gland, adrenal gland, urethra, prostate, penis, testes, ureter, bladder, kidney or liver; rectal cancer, cancer of the anal region, carcinomas of the fallopian tubes, endometrium, cervix, vagina, vulva, renal pelvis, renal cell, sarcoma of soft tissue, myxoma, rhabdomyoma, fibroma, lipoma, teratoma, cholangiocarcinoma, hepatoblastoma, angiosarcoma, hemangioma, hepatoma, fibrosarcoma, chondrosarcoma, myeloma, chronic or acute leukemia, lymphocytic lymphomas, primary CNS lymphoma, neoplasms of the CNS, spinal axis tumors, squamous cell carcinomas, synovial sarcoma, malignant pleural mesotheliomas, brain stem glioma, pituitary adenoma, meningioma, bronchial adenoma, chondromatous hanlartoma, inesothelioma, Hodgkin's Disease, brain (gliomas), glioblastomas, astrocytomas, glioblastoma multiforme, Bannayan-Zonana syndrome, Cowden disease, Lhermitte-Duclos disease, Wilm's tumor, Ewing's sarcoma, Rhabdomyosarcoma, ependymoma, medulloblastoma, melanoma, ovarian, pancreatic, adenocarcinoma, ductal madenocarcinoma, adenosquamous carcinoma, small cell lung cancer, acinar cell carcinoma, glucagonoma, insulinoma, prostate, sarcoma, osteosarcoma, giant cell tumor of bone, thyroid, lymphoblastic T cell leukemia, chronic myelogenous leukemia, chronic lymphocytic leukemia, hairy-cell leukemia, acute lymphoblastic leukemia, acute myelogenous leukemia, chronic neutrophilic leukemia, acute lymphoblastic T cell leukemia, plasmacytoma, Immunoblastic large cell leukemia, mantle cell leukemia, multiple myeloma, megakaryoblastic leukemia, multiple myeloma, acute megakaryocyte leukemia, pro myelocytic leukemia, erythroleukemia, malignant lymphoma, hodgkins lymphoma, non-hodgkins lymphoma, lymphoblastic T cell lymphoma, Burkitt's lymphoma, follicular lymphoma, neuroblastoma, bladder cancer, urothelial cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor) and testicular cancer.

In some embodiments, the cancer is selected from the group consisting of small cell lung cancer, non-small cell lung cancer, mesothelioma, meningioma, and triple negative breast cancer.

Another aspect of the present disclosure provides a pharmaceutical composition for the treatment of cancer, comprising a stimulator of interferon signaling, a heparin polysaccharide, and a pharmaceutically acceptable excipient. In some embodiments, the heparin polysaccharide has reduced anticoagulant activity. In some embodiments, the heparin polysaccharide is at least one of desulfated and N-acetylated. In some embodiments, the heparin polysaccharide is at least one of N-desulfated and O-desulfated. In some embodiments, the heparin polysaccharide is at least one of 2-O desulfated, 3-O desulfated, and 6-O desulfated. In some embodiments, the heparin polysaccharide comprises a glycol-split monomer. In some embodiments, the heparin polysaccharide is low molecular weight heparin. In some embodiments, the heparin polysaccharide lacks a unique pentasaccharide sequence, wherein the unique pentasaccharide sequence has the following general structure:

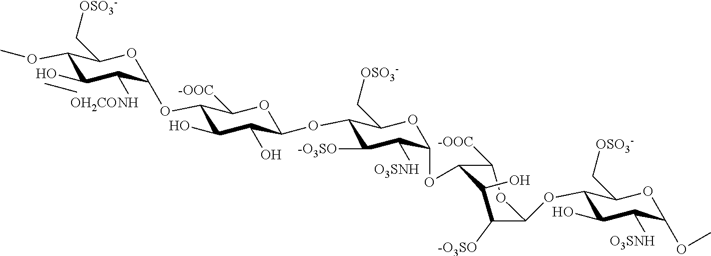

In some embodiments, the stimulator of interferon signaling is selected from the group consisting of interferon alpha, interferon beta, STING agonists, TLR agonists, and oncolytic viruses. In some embodiments, when the stimulator of interferon signaling is a STING agonist, it is selected from the group consisting of cyclic GMP-AMP (cGAMP), ganciclovir, ADU-S100, and CMA. In some embodiments, the pharmaceutically acceptable excipient is water or saline.

In some embodiments of the present disclosure, the heparin polysaccharide in the method or pharmaceutical composition does not comprise a synthetic pentasaccharide. In some embodiments of the present disclosure, the heparin polysaccharide in the method or pharmaceutical composition does not comprise fondaparinux.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. For purposes of clarity, not every component may be labeled in every drawing. It is to be understood that the data illustrated in the drawings in no way limit the scope of the disclosure. In the drawings:

FIG. 1 includes plots showing that heparin enhances STING agonist activity in cancer cells. Human and mouse cancer cell lines were treated with ADU S-100 (50 μM unless otherwise noted)+/−heparin at a concentration of 10 μg/mL (human cells) or 1 μg/mL (mouse cells) for 24 hours prior to conditioned media collection for CXCL10 ELISA. SCLC=small-cell lung cancer. NSCLC=non-small-cell lung cancer. TNBC=triple negative breast cancer. GBM=glioblastoma. ANOVA p<0.001 for all figures. *p<0.05 **p<0.01 ***p<0.001 ****p<0.0001 by Bonferroni corrected pairwise comparison.

FIG. 2 includes plots showing that heparin enhances STING agonist activity. FIG. 2A includes plots showing human lung fibroblasts (hLFB) and H69-mesenchymal (H69M) small cell lung cancer (SCLC) cells treated with 2,3-cGAMP 1 μg/mL+/−heparin 1 μg/mL for 24 hours prior to CXCL10 qPCR and collection of conditioned media for C—X—C motif chemokine 10 (CXCL10) ELISA (enzyme-linked immunosorbent assay). FIG. 2B includes plots showing human and mouse immortalized cell lines treated with ADU S-100 (50 μM unless otherwise noted), 2′3′-cGAMP (cGAMP; 10 μg/mL), or IFN-beta (IFNb 1 ng/mL)+/−heparin at a concentration of 10 μg/mL (human cells) or 1 μg/mL (mouse cells) for 24 hours prior to conditioned media collection for CXCL10 ELISA. THP1=differentiated macrophages. hLFB=human lung fibroblasts. MEF=mouse embryonic fibroblasts. HUE=human umbilical endothelial cells. ANOVA p<0.001 for all figures. *p<0.05 ****p<0.0001 by Bonferroni corrected pairwise comparison.

FIGS. 3A to 3B include plots showing that heparin dose-dependently enhances STING agonist effects across compounds. FIG. 3A shows CXCL10 ELISA results from conditioned media of 631M/RPPM mouse SCLC cells after 24-hour treatment at the indicated doses of STING agonists+/−heparin at a concentration of 1 μg/mL. FIG. 3B shows CXCL10 ELISA results from conditioned media of H69M human SCLC cells after 24-hour treatment at the indicated doses of the STING agonist 2′3′-cGAMP+/−heparin at the indicated concentrations. FIG. 3C includes plots showing a dose course of the STING agonist ADU S-100 in Benign-Meningioma-1 (BEN-MEN-1) meningioma cells with the doses shown in μM+/−10 μg/mL heparin, as well as treatment with STING agonists 2,3-cGAMP, ADU-S100, and 10-(carboxymethyl)-9(10H)acridone (CMA) in RPPM primary mouse SCLC cells (described in Material and Methods section). FIG. 3D includes a plot of showing the time course data for BenMen 1 cells with treatment for 3 and 6 days. The data reflects 24 hours treatment prior to collection of conditioned media for CXCL10 ELISA FIG. 3E includes plots showing RPPM mouse SCLC cells treated with 1 μg/mL 2,3-cGAMP and 1 μg/mL unfractionated heparin, low-molecular weight heparin (LMWH), heparin pentasaccharide fondaparinux, 6-desulfated heparin, chondroitin sulfate (CS)+/−the Janus kinase/signal transducers and activators of transcription (JAK/STAT) inhibitor ruxolitinib (ruxo 1 μg/mL) for 24 hours prior to CXCL10 ELISA. H69M human SCLC cells treated with 10 μg/mL 2,3-cGAMP+/−heparin 10 μg/mL or desulfated heparins 2-O desulfated (2DES), N-desulfated (NDES), and 6-O desulfated (6DES) 24 hours prior to CXCL10 ELISA. All panels reflect 24 hours treatment prior to collection of conditioned media for CXCL10 ELISA.

FIGS. 4A-D include diagrams showing that heparin increases STING agonist uptake and activation of downstream signaling. FIG. 4A includes immunofluorescent images of fixed hLFB cells after 24 hours of treatment with cyanine-5 (Cy5) labeled 2,3-cGAMP 1 μg/mL+/−heparin 1 μg/mL. The staining represents actin phalloidin, DAPI (4′,6-diamidino-2-phenylindole), and Cy5-labeled cGAMP. FIG. 4B includes a western blot for STING pathway components in BEN-MEN-1 meningioma cells treated for 72 hours with 50 μM ADU+/−heparin 10 μg/mL and MRT TANK-binding kinase-1 (TBK1) inhibitor 5 μM. FIG. 4C includes plots showing CXCL10 ELISA after 24 hours treatment with 2,3-cGAMP (1 μg/mL) or ADU S-100 (50 μM unless otherwise indicated)+/−heparin (1 μg/mL in RPPM or 10 μg/mL in MS428), MRT TBK1 inhibitor 1 μM, or ruxolitinib JAK/STAT inhibitor (“ruxo”; ruxolitinib) 1 μM in RPPM mouse SCLC and MS428 human mesothelioma cell lines. FIG. 4D includes a plot showing the qPCR for Programmed death-ligand 1 (PD-L1) after 24 hours treatment 50 μM ADU+/−heparin 10 μg/mL and MRT TBK1 inhibitor in BEN-MEN-1 meningioma cells. ANOVA p<0.001 for all graphs. *p<0.05 ****p<0.0001 by Bonferroni corrected pairwise comparison.

FIGS. 5A to 5B include plots showing heparin increases STING agonist suppression of cancer cell growth in vitro. FIG. 5A shows the results of a cell-titer glow proliferation assay with H69M human SCLC cells after 24 hours of treatment with 50 μM ADU+/−heparin (10 μg/mL) and RPPM mouse SCLC cells after 48 hours of treatment with 50 μM ADU+/−heparin 1 μg/mL. ANOVA p<0.001. *p<0.05 **p<0.01 by Bonferroni corrected pairwise comparison. FIG. 5B shows the results of a cell-titer glow proliferation assay in BEN-MEN-1 meningioma cells after a 24-hour treatment with 50 μM ADU+/−heparin 10 μg/mL. *p<0.05 by 2-tailed Student's t-test.

FIG. 6 includes plots showing IL-8 levels from Luminex cytokine profiling after a 24-hour treatment with 50 μM ADU+/−heparin 10 μg/mL and MRT TBK1 inhibitor 5 μM in MS428 meningioma cells and H69M SCLC cells. Also shown is IL-8 ELISA confirmation of decreased growth promoting IL-8 after heparin treatment (10 μg/mL if unlabeled, 10=10 μg/mL; 1=1 μg/mL) with or without ADU 50 μM or 2,3-cGAMP 1 μg/mL ANOVA p<0.01. *p<0.05 **p<0.01 by Bonferroni-corrected pairwise comparison.

FIG. 7 includes a schematic showing a glycol split monomer formed by cleavage of the bond between two hydroxyl groups in the antithrombin-binding domain taken from Poli, Maura, et al. (Blood 123.10 (2014): 1564-1573).

FIGS. 8A to 8D include plots and Western blots showing heparin enhances type I interferon effects but not interferon gamma effects. FIG. 8A and FIG. 8B show ELISA results for CXCL10 in the media of B16F10 mouse melanoma cells treated for 24 hours with interferon alpha (IFNa), interferon beta (IFNb) or interferon gamma (IFNg) 5 ng/ml+/−heparin (1 μg/mL) (FIG. 8A), or for CXCL10 in the media of Lewis Lung Carcinoma (LLC) mouse non-small-cell lung cancer cells treated for 24 hours with interferon alpha (IFNa), interferon beta (IFNb) or interferon gamma (IFNg) 5 ng/ml+/−heparin (5 μg/mL) (FIG. 8B). ANOVA p<0.0001. *p<0.05 ****p<0.0001 by Bonferroni corrected pairwise comparison. FIG. 8C shows a Western blot for pSTAT1 and beta-actin load control after treatment with interferons+/−heparin (1 μg/mL for B16F10 unless otherwise noted). FIG. 8D shows a Western blot for pSTAT1 and beta-actin load control after treatment with interferons+/−heparin (1 μg/mL for H69M unless otherwise noted).

FIGS. 9A to 9B include plots showing heparin-IFNb effects are dose dependent. FIG. 9A shows the results of a CXCL10 ELISA from conditioned media of B16F10 mouse melanoma cells after 24 hours treatment at the indicated doses of IFNb+/−heparin at the indicated doses. ANOVA p<0.0001 figures, ****p<0.0001 by Bonferroni corrected pairwise comparison. FIG. 9B shows the results of a CXCL10 ELISA from conditioned media of B16F10 mouse melanoma cells after 24 hours treatment at the indicated doses of IFNb+/−heparin (1 μg/mL). ANOVA p<0.0001 figures, ****p<0.0001 by Bonferroni corrected pairwise comparison.

FIGS. 10A to 10B includes plots showing modified heparins also enhance IFNb and STING effects. FIG. 10A shows the results of aCXCL10 ELISA from conditioned media of B16F10 mouse melanoma cells after 24-hour treatment at the indicated doses of IFNb+/−heparins (1 μg/mL) including unfractionated heparin, low-molecular weight heparin (LMWH), 2- and 6-, and N-desulfated heparin (2DES, 6DES, NDES), heparin pentasaccharide fondaparinux, as well as controls including chondroitin sulfate (CS), rivaroxaban. FIG. 10B shows the result of a CXCL10 ELISA from conditioned media of RPPM mouse SCLC cells after 48 hours treatment at the indicated doses of 2′3′-cGAMP (1 μg/mL)+/−heparins (all at a concentration of 1 μg/mL except CS at 10 μg/mL) and the JAK/STAT inhibitor ruxolitinib (ruxo 1 μg/mL). ANOVA p<0.0001 for both figures. *p<0.05 **p<0.01 ****p<0.0001 by Bonferroni corrected pairwise comparison.

FIGS. 11A to 11B include plots showing heparin enhances CXCL10 downstream of multiple inflammatory stimuli. FIG. 11A shows the results of a CXCL10 ELISA from conditioned media of B16F10 mouse melanoma cells after 4 hours transfection with 1 g Poly(dA:dT) or Poly(I:C) followed by treatment with heparin (1 μg/mL) or control for 24 hours. FIG. 11B shows the results of a CXCL10 ELISA from conditioned media of H196 human SCLC cells after 4 hours transfection with 1 g Poly(dA:dT) followed by treatment with heparin (10 μg/mL) or control for 24 hours. ANOVA p<0.0001 for all figures. *p<0.05 **p<0.01 by Bonferroni corrected pairwise comparison.

FIGS. 12A to 12C includes plots showing heparin requires an upstream stimulus, and does not enhance ISRE binding. FIG. 12A shows the results of a CXCL10 ELISA from conditioned media of B16F10 mouse melanoma cells after 24 hours of treatment with IFNb (1 ng/mL)+/−heparin (1 μg/mL) and either 0.5 μM MRT67307 (MRT) or 0.5 μM Ruxolitinib (ruxo). FIG. 12B shows the results of a CXCL10 ELISA from conditioned media of B16 Blue cells purchased from Invivogen treated with IFNb (500 μg/mL), ADU-S100 (50 μM)+/−heparin (5 μg/mL). ANOVA p<0.0001. ****p<0.0001 by Bonferroni corrected pairwise comparison. FIG. 12C shows the results from the same samples with an ISRE chromogenic reporter assay used according to manufacturer's instructions. ANOVA p<0.0001. ****p<0.0001 by Bonferroni corrected pairwise comparison.

FIGS. 13A to 13C includes plots showing heparin enhances CXCL10 release from cells treated with IFNb. FIG. 13A shows the results of a CXCL10 PCR from of B16F10 mouse melanoma cells after a time course of treatment with IFNb (500 μg/mL)+/−heparin (5 μg/mL). FIG. 13B shows the results of a CXCL10 ELISA from conditioned media of B16F10 mouse melanoma cells after a time course of treatment with IFNb (500 μg/mL)+/−heparin (5 μg/mL). FIG. 13C shows the results of aCXCL10 ELISA from conditioned media and cell lysates of B16F10 mouse melanoma cells after six hour treatment with IFNb (5 ng/mL)+/−heparin (1 μg/mL) as well as Golgi-Stop from BD biosciences (1 μL). ANOVA p<0.0001. ***p<0.001 ****p<0.0001 by Bonferroni corrected pairwise comparison.

FIGS. 14A to 14C includes plots showing heparin enhances CXCL10 release from cells treated with STING agonists. FIG. 14A shows the results of a CXCL10 ELISA from conditioned media and cell lysates of B16F10 mouse melanoma cells after six-hour treatment with ADU-S100 (50 μM)+/−heparin (5 μg/mL). FIG. 14B shows the results of a CXCL10 ELISA from conditioned media and cell lysates of B16F10 mouse melanoma cells after six-hour treatment with ADU-S100 (50 μM)+/−heparin (1 μg/mL) as well as Golgi-Stop or Golgi-Plug from BD biosciences (0.5 μL). FIG. 14C shows the results of a CXCL10 ELISA from conditioned media and cell lysates of MS428 human mesothelioma cells after twelve-hour treatment with ADU-S100 50 μM+/−heparin 10 μg/mL as well as Golgi-Stop from BD biosciences (0.5 μL per manufacturer's instructions). ANOVA p<0.0001. *p<0.05 **p<0.01 by Bonferroni corrected pairwise comparison.

FIG. 15 includes diagrams and plots showing Heparin enhances cytokine release. CXCL10 ELISA from conditioned media and cell lysates of MS428 human mesothelioma cells after six-hour treatment with ADU-S100 50 μM (top panel, right of image), followed by media change and subsequent treatment with control or heparin 10 μg/mL as well as Golgi-Plug (GP) from BD biosciences (0.5 μL per manufacturer's instructions). One-way ANOVA p<0.0001. *p<0.05, **p<0.01 ****p<0.0001 by Bonferroni corrected pairwise comparison.

FIG. 16 includes plots showing that Heparin must be internalized to have an effect. CXCL10 ELISA from conditioned media of B16F10 mouse melanoma cells after six hour (left panel) or twenty-four-hour (right panel) treatment with ADU-S100 50 μM+/−heparin (5 μg/mL for 6-hour treatment, 1 μg/mL for twenty-four-hour treatment), as well as heparin-Sepharose beads (HEP-SEPH; Abcam) per manufacturer's instructions at equivalent doses to unfractionated heparin. Ttest: ***p<0.001, ****p<0.0001.

FIGS. 17A to 17D includes images showing that Heparin does not co-localize with Golgi markers. Immunofluorescence of MS428 human mesothelioma cells were grown in chamber slides (CellTreat) and treated for six-hours with GFP-labeled heparin (invitrogen) at μg/mL prior to PFA fixing, methanol permeabilization, and staining with Golgin 97 antibody from Cell Signaling Technology (13192) per manufacturer's instructions at a dilution of 1:50 overnight, followed by goat anti-Rabbit IgG (H+L) Cross-Adsorbed Secondary Antibody, Alexa Fluor 555 (Invitrogen A21428) for 1-hour at 1:1000. Slides were mounted with anti-fade+DAPI and imaged using Z-stack on a Nikon Eclipse 80i microscope. Colocalization was quantified from three high power fields and background from GFP-Heparin treated cells without Golgin antibody was subtracted before calculating the Pearson Correlation co-efficient (r).

FIGS. 18A to 18D includes images showing that Heparin co-localizes at some endosomes. Immunofluorescence of MS428 human mesothelioma cells were grown in chamber slides (CellTreat) and treated for six-hours with GFP-labeled heparin (invitrogen) at μg/mL prior to PFA fixing, methanol permeabilization, and staining with Syntaxin 6 antibody from Cell Signaling Technology (2869) per manufacturer's instructions at a dilution of 1:50 overnight, followed by goat anti-Rabbit IgG (H+L) Cross-Adsorbed Secondary Antibody, Alexa Fluor 555 (Invitrogen A21428) for 1-hour at 1:1000. Slides were mounted with anti-fade+DAPI and imaged using Z-stack on a Nikon Eclipse 80i microscope. Colocalization was quantified from three high power fields and background from GFP-Heparin treated cells without Syntaxin 6 antibody was subtracted before calculating the Pearson Correlation co-efficient (r).

FIGS. 19A to 19B includes diagrams showing that heparin alters the release of multiple cytokines after STING agonist treatment. FIG. 19A show a Luminex cytokine array after 24-hour treatment with 50 μM ADU+/−heparin (10 μg/mL)+/−the MRT TBK1 inhibitor (5 μM) in H196 SCLC and MS428 mesothelioma cells, demonstrating an increase in T cell recruiting and growth suppressive cytokines such as CXCL10 and CCL5 and a decrease in growth promoting cytokines such as IL-8 with the addition of heparin to ADU, which is reversed by MRT. L2FC=LOG2 fold change. FIG. 19B shows a diagram of signaling pathways implicated and heparin's effects.

FIGS. 20A to 20F includes plots showing that ex vivo treatment confirms that heparin enhances CXCL10 release. Treatment of patient-derived organotypic spheroids (PDOTs) for 1-6 days with ADUS100 (50 μM)+/−heparin 10 μg/mL prior to collection of conditioned media for CXCL10 ELISA. IFNb at a concentration of 1 ng/mL. PDOTs were as per Jenkins et al., Cancer Discovery, 2018. The samples were Mesothelioma patient specimens. ANOVA p<0.0001. *p<0.05, **p<0.01, ***p<0.001, ****p<0.0001 by Bonferroni corrected pairwise comparison.

FIG. 21 includes graphs of in vivo data from Immune cell profiling from the 631 RPP mouse SCLC syngeneic model in BL6J. One tumor from each group was collected 3 days after intra-tumoral (IT) injection and processed using a Miltenyi dissociation kit prior to flow cytometry using a previously published panel of immune-cell antibodies.

### DETAILED DESCRIPTION OF THE INVENTION

The present disclosure relates to the unexpected discovery that the coadministration of a heparin polysaccharide and a stimulator of interferon genes (STING) agonist (e.g., cyclic guanosine monophosphate-adenosine monophosphate (cGAMP)) leads to (i) enhanced activity of the STING agonist and (ii) enhanced delivery of the STING agonist into the cell, and (iii) localization of the heparin and the STING agonist in the correct (targeted) place. The enhanced activity of the STING agonists by the coadministration of heparin resulted in an increase interferon-stimulated gene expression including CXCL10, IFITM1, and IFI27. However, no downstream activation of pTBK1 or pIRF3 occurred following the addition of heparin to the STING agonists cGAMP and ADU-S100. Type I interferon signaling provided a robust and dose-dependent increase in CXCL10 production in the presence of heparin.

Thus, the present disclosure provides, inter alia, methods for the treatment of cancer by coadministering a heparin polysaccharide and a stimulator of interferon signaling (such as interferon alpha, interferon beta, or a STING agonist). While there is literature suggesting the use of certain forms of heparin in the treatment of cancer, Applicant is not aware of any literature suggesting the combination of heparin and a stimulator of interferon signaling having a synergistic effect on cells.

### Interferon Signaling

Type I interferon signaling (alpha and beta) enhances immune cell recruitment and activation to promote anti-tumor immunity. Type I interferons have been used clinically to treat melanoma, myeloproliferative disorders including multiple myeloma, certain types of lymphoma, prostate cancer, and renal cell carcinoma. Activators or stimulators or type I interferon signaling, including oncolytic viruses, TLR agonists, STING agonists, and other mechanisms of immunogenic cell death, all enhance type I interferon signaling to promote anti-tumor immunity.

The treatment with stimulators or interferon signaling and heparin resulted in enhanced CXCL10 levels in the cell culture media in vitro. Disclosed herein are methods of use for heparin, and its desulfated variants, as a therapeutic in human tumors to enhance the activity of stimulators or interferon signaling, including but not limited to interferon alpha, interferon beta, and STING agonists (including, but not limited to, 2,3-cGAMP, ADU-S100, and ganciclovir) and use of its ability to synergize with check point inhibitors such as PD-1 inhibitors and programmed death-ligand 1 (PD-L1) inhibitors. Herein, it is demonstrated that this enhancement promotes anti-tumor immune activity and tumor regression.

Disclosed herein, are methods and compositions for coadministering a stimulator of interferon signaling and a heparin polysaccharide to a subject having cancer. It was found that the coadministration of heparin polysaccharide and a stimulator of interferon signaling increases the amount of CXCL10 release by the cells (e.g., cancer cells) relative to the administration of heparin alone or stimulator of interferon signaling alone. As used, the terms “heparin alone” and “stimulator of interferon signaling alone” refer to the treatment/administration of either heparin or stimulator of interferon signaling (e.g., interferon alpha, interferon beta, cGAMP, ADU S-100), respectively, without administering the other.

### STING Activity

Stimulator of interferon genes (STING; also referred to as transmembrane protein 173 (TMEM173)) functions as an adaptor protein downstream of intracellular DNA sensing by the enzyme cyclic GMP-AMP synthase (cGAS). cGAS produces the second messenger cGAMP, which recruits STING to activate TANK-binding kinase-1 (TBK1) and Interferon Regulatory Factor 3 (IRF3), leading to upregulation of the chemokine C—X—C Motif Chemokine Ligand 10 (CXCL10) and T-cell recruitment. cGAMP is a cyclic dinucleotide that can be released from tumor cells to act in a paracrine manner. cGAMP and other STING agonists have shown therapeutic promise in preclinical models of human cancer via activation of innate immune signaling to enhance cytotoxic T cell activity and sensitize to programmed cell death protein 1 (PD-1) inhibitors. However, the response to STING agonists (e.g., cGAMP) has been limited by cellular uptake and systemic activity, requiring intratumoral injections at high doses. Herein, increased STING activity, measured by phospho-TBK1, specifically in endothelial cells of human tumors (and not in normal vasculature endothelial cells) was observed. Unexpectedly, it was observed that endothelial cell culture media enhances CXCL10 production in human lung fibroblasts (hLFBs) after treatment with a low dose (e.g., 1 μg/mL) of STING agonist (e.g., 2,3-cGAMP). This was an unexpected finding, because these cultured fibroblasts do not typically respond to this low dose of cGAMP.

As used herein, “STING activity” refers to the activation of STING signaling pathways. Without being bound by theory or mechanism, the activation of the STING signaling pathway stimulates TBK1 activity to phosphorylate IRF3 or Signal transducer and activator of transcription 6 (STAT6). Phosphorylated IRF3s and STAT6s dimerize then enter the nucleus where they stimulate interferon related genes (e.g., Interferon Beta 1 (IFNB), C—C Motif Chemokine Ligand 2 (CCL2), C—C Motif Chemokine Ligand 20 (CCL20), C—X—C Motif Chemokine Ligand 10 (CXCL10), and C—C Motif Chemokine Ligand 5 (CCL5)).

Methods to measure STING activity are known in the art. Non-limiting examples of methods that can be used to measure include quantitative PCR (qPCR) and enzyme-linked immunosorbent assay (ELISA) to measure the expression of genes or concentration of proteins/cytokines/chemokines downstream the STING signaling pathway. For example, the STING activity in cells can be measured using qPCR to determine the expression levels of CXCL10. Alternatively, the STING activity in cells can be measured using ELISA to detect concentration of CXCL10.

### Sting Agonists

While STING agonists have shown promise in animal models, recent early phase clinical data has been disappointing. One potential barrier to efficacy is the requirement for intratumoral injection, as well as the inability for cyclic dinucleotides (many of these compounds are structurally similar to cGAMP) to cross the cell membrane and activate STING. A poor response to cGAMP in vitro was observed across human cancer cell lines despite robust activity in mouse models. The present disclosure, inter alia, provides methods for coadministering a heparin polysaccharide and a STING agonists to increase response and activity of STING agonists in human cancer cells. These methods allow for efficient pathway activation and potentially simplify drug delivery. The model systems tested herein have previously been shown to predict response to PD1 inhibitors or PDL-1 inhibitors, suggesting potential synergy that would help increase response rates in patients.

Non-limiting examples of STING agonists that can be used in methods of the present disclosure include ganciclovir, cyclic dinucleotides (CDNs): for example, ADU-S100 (MIW-815), cGAMP, cGAMP bisphosphorothioate, 2′3′-cGAMP, c-di-AMP, c-di-GMP (cyclic diguanylate), 3′3′-cGAMP, and 3′2′-cGAMP, xanthenone derivatives such as DMXAA, and the like; c-AIMP; (3′,2′) c-AIMP; (2′,2′)c-AIMP; (2′,3′) c-AIMP; c-AIMP(S); c-(dAMP-dlMP); c-(dAMP-2′FdlMP); c-(2′FdAMP-2′FdlMP); (2′,3′)c-(AMP-2′FdlMP); c-[2′FdAMP(S)-2′FdlMP(S)]; c-[2′FdAMP(S)-2′FdlMP(S)](POM)2; Rp,Rp dithio 2′,3′ c-di-AMP (e.g., Rp,Rp dithio c-[A(2′,5′)pA(3′,5′)p] or a cyclic dinucleotide analog thereof); c-[G(2′,5′)pG(3′,5′)p]; c-[G(2′,5′)pA(3′,5′)p]; and 2′-O-propargyl-cyclic-[A(2′,5′)pA(3′,5′)p] (2′-0-propargyl-ML-CDA). Non-limiting examples of STING agonists include flavonoids: flavone acetic acid (FAA), 10-(carboxymethyl)-9(10H)acridone (CMA), 5,6-Dimethylxanthenone-4-acetic acid (DMXAA; Vadimezan), methoxyvone, 6, 4′-dimethoxyflavone, 4′-methoxyflavone, 3′, 6′-dihydroxyflavone, 7, 2′-dihydroxyflavone, daidzein, formononetin, retusin 7-methyl ether, xanthone, or any combination thereof. Non-limiting examples of STING agonists include cyclic dinucleotide (CDN) derivatives and locked-nucleic acid cyclic dinucleotides (LN-CDN). Additional examples of STING agonists include SB-11285, MK-1454, SR-8291, AdVCA0848, GSK-532, SYN-STING, MSA-1, and SR-8291. Non-limiting examples of cyclic di-nucleotides are described in Patent Applications WO 2014093936, WO 2014189805, WO 2013185052, US 20140341976, WO 2015077354, PCT/EP2015/06228, US20180230171 and GB 1501462, the entire disclosures of which are incorporated herein by reference. Non-limiting examples of STING agonists are is described in US20170158772, US20150056224, US20160287623, U.S. Ser. No. 10/106,574, U.S. Ser. No. 10/045,961, US20190031708, U.S. Pat. No. 1,004,711, US20180230177, US20180230115, US20140329889, US20160331810, US20190185511, WO2017186711 and WO2016145102, the entire disclosures of which are incorporated herein by reference. As used herein, “cyclic dinucleotides” can include salts of those described herein.

As used herein, the term “cyclic dinucleotide” can refer to a single-phosphate nucleotide with a cyclic bond arrangement between the sugar and phosphate groups. Cyclic dinucleotides (CDN) can include isoforms (e.g., tautomers). In nature, bacteria and other microbes produce CDN, for example c-diGMP, c-diAMP and c-diGAMP, and release them into their hosts. Metazoans synthesize also CDN (e.g., 2′3′-cGAMP). They can be obtained using any suitable method (e.g., chemical synthesis from nucleoside derivatives, in vitro synthesis, e.g., from recombinant purified cGAMP synthase).

In some embodiments of the present disclosure, the STING agonist is selected from the group consisting of cyclic GMP-AMP (cGAMP), ganciclovir, and ADU-S100.

CGAMP is a cyclic dinucleotide that is synthesized by metazoans, The structure of an exemplary cGAMP is shown below:

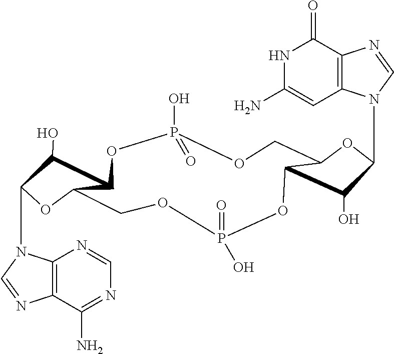

Ganciclovir is a STING agonist that is also used as an anti-viral medication. The structure of an exemplary ganciclovir is shown below:

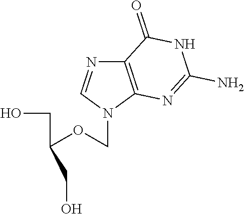

ADU-S100 (MIW815) is a synthetic cyclic dinucleotide that functions as a STING agonist. The structure of an exemplary ADU-S100 is shown below:

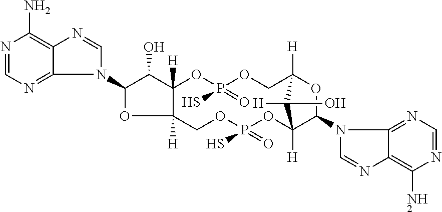 [SMILES: Nc1ncnc2c1ncn2[C@@H]1O[C@@H]2CO[P@](=O)(S)O[C@@H]3C(O)[C@@H](CO[P@@](=O)(S)O[C@H]2[C@H]1O)O[C@H]3n1cnc2c(N)ncnc21]

10-(carboxymethyl)-9(10H)acridone (CMA or Cridanimod) is a flavonoid STING agonist that directly binds to STING and has been shown to trigger a strong antiviral response through the TBK1/IRF3 route. CMA triggers type I IFN response in murine macrophages. The structure of an exemplary CMA is shown below:

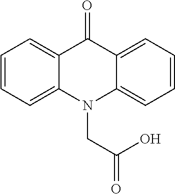 [SMILES: O=C(O)Cn1c2ccccc2c(=O)c2ccccc21]

In some embodiments of the present disclosure more than one type of STING agonist is administered with the heparin polysaccharide.

### Innate Immune Therapies

Stimulators of interferon signaling and STING agonists are examples of innate immune therapies. The present disclosure provides that treatment with stimulators of interferon signaling and STING agonists along with heparin results in enhanced release of cytokines from the cells, including but not limited to CXCL10 levels. However, other innate immune therapies can result in or require release of CXCL10 from cells. Accordingly, other innate immune therapies that result in or require release of CXCL10 from cells should also be enhanced by coadministration with heparin. For example, T cells can secrete CXCL10, so agents that stimulate CD8 T cell activation could be enhanced by the addition of heparin. Moreover, 4-1BB and OX40 agonists could be coadministered with heparin because use of these agents has been shown to increase CXCL10. In addition, tumor vaccines, whether T-cell or dendritic cell, and adoptive cell transfer lead to increases in CXCL10, and so could also be co-administered with heparin. Innate immune therapies are known in the art, for example, as disclosed in Saibil and Ohashi, Targeting T Cell Activation in Immune-Oncology, Current Oncology, 27(S2):98-105 (2020), the contents of which are incorporated by reference in their entirety.

### Heparin

The methods of the present disclosure include administering (i.e. coadministering) a therapeutically effective amount of a STING agonist and a therapeutically effective amount of a heparin polysaccharide to a subject. As used herein, the term “heparin polysaccharide” includes means molecules having a heparin backbone and includes heparin fragments. Non-limiting examples of molecules that can be considered a heparin polysaccharide include: unfractionated heparin; low molecular weight heparins such as enoxaparin, dalteparin, tinzaparin, and fondaparinux; heparin derivatives including, but not limited to, heparin sulfate, heparinoids, heparin-based compounds, heparin derivatized with hydrophobic materials and earth metal salts of heparin such as, for example, sodium heparin, potassium heparin, lithium heparin, calcium heparin, and magnesium heparin; high molecular weight heparins; heparin analogues; and synthetic heparins (e.g., fondaparinux). Non-limiting examples of molecules that can be considered a heparin polysaccharide include: Fragmin, Innohep (tinzaparin), Lovenox (enoxaparin), Heparin Sodium, Monoject Prefill Advanced (heparin flush), Orgaran (danaparoid), and PosiFlush (heparin flush).

Heparin is a sulfated polysaccharide composed of repeating disaccharide units (D-glucosamine and uronic acid (glucuronic acid or iduronic acid)) sulfated at the 3-O, 6-O, and N sites of glucosamine and the 2-O site of glucuronic acid. Heparin compositions are a heterogeneous mixture of polysaccharide chains that vary in length and therefore molecular weight. There are various forms of heparin (e.g., unfractionated heparin, low molecular weight heparin). Low molecular weight heparin (LMWH) can be prepared from unfractionated heparin by enzymatic or depolymerization techniques. Non-limiting examples of low molecular weight heparins are shown in the table below:

Low molecular weight heparin preparations

| Preparation | Method of preparation | Molecular weight |
|---|---|---|
| 1. Ardeparin | Peroxidative depolymerisation | 6000 |
| 2. Dalteparin | Nitrous acid depolymerisation | 6000 |
| 3. Enoxaparin | Alkaline depolymerisation | 4200 |
| 4. Nadroparin | Nitrous acid depolymerisation | 4500 |
| 5. Reviparin | Nitrous acid depolymerisation | 4000 |
| 6. Tinzaparin | Heparinase digestion | 4500 |

The range of molecular weight in a heparin mixture can be anywhere from about 1800 to 30,000 Da. Most commercially-available heparin mixtures include molecules ranging from 12 to 15 kDa. In addition, these mixtures may also comprise heparin fragments that are a lower molecular weight. Low molecular weight heparin is known to have an average molecular weight of about 5000.

The sulfation sites in heparin molecules aid in the binding of heparin to antithrombin (also referred to as antithrombin III) and contribute to the anticoagulation activity of heparin. Antithrombin functions by accelerating the coagulation ability of enzymes thrombin (factor IIA), factor Xa, and factor IXA. The anticoagulant activity of heparin molecules is mainly due to their affinity to antithrombin, specifically to a pentasaccharide sequence known as the antithrombin III binding site (AT-bs; also referred to as the antithrombin III binding motif/sequence). Not all heparin molecules have the AT-bs pentasaccharide sequence. The sequence of the AT-bs is GclNAc6SO3-GlcA-GlcNSO3-6SO3-IdoA2SO3-GlcNSO3.6SO3 and it has the following structure:

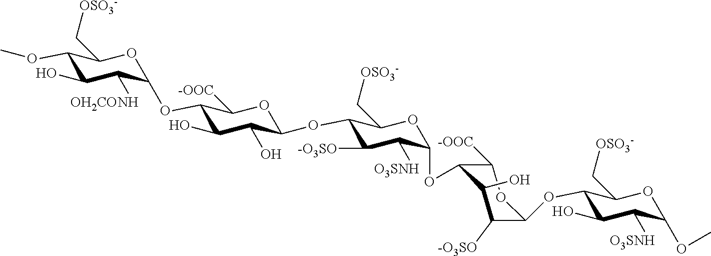

To effectively bind to thrombin, an AT-bs-containing heparin molecule must be of adequate length to bind to both antithrombin and thrombin. The threshold length for this binding is 18 saccharide units (equivalent to a molecular weight of about 5000). It is estimated that less than half of LMWH chains exceed this threshold length. Heparin chains that are less than 5000 in molecular weight may still have anticoagulant activity due to their ability to bind to antithrombin and factor Xa, thereby inactivating factor Xa.

In some embodiments, more than one type of heparin polysaccharide (e.g., unfractionated heparin, LMWH) is administered with the STING agonist. The more than one type can be a combination of any two of the heparins described above.

### Synthetic Pentasaccharides

In some embodiments of the present disclosure, the heparin polysaccharide is a synthetic pentasaccharide, also referred to as synthetic heparins, (e.g., fondaparinux, idraparinux, etc.). In some embodiments, the heparin polysaccharide is not a synthetic pentasaccharide (e.g., fondaparinux, idraparinux, etc.). Many of these synthetic heparins are synthesized using the AT-bs backbone. In some embodiments, synthetic heparins may be used to refer to the synthetic heparins (e.g., fondaparinux, idraparinux), their analogues, their derivatives (e.g., idrabiotaparinux), and/or salts thereof (e.g., sodium salt derivative). In some embodiments, the heparin polysaccharides of the present disclosure do not include these.

Fondaparinux is a synthetic pentasaccharide factor Xa inhibitor. Its structure is based on the pentasaccharide sequence that makes up the minimal antithrombin (AT) binding site (the AT-bs). Without being bound by theory or mechanism, in plasma, fondaparinux selectively binds to antithrombin, catalyzes factor Xa inhibition, and thereby inhibits thrombin generation. Idraparinux is an analogue of fondaparinux binding with high affinity to antithrombin. It is a long-acting inhibitor, as opposed to fondaparinux, which is a short acting inhibitor.

### Reduced Anticoagulant Activity

In some embodiments of the present disclosure, the heparin polysaccharide that is coadministered with the stimulator or interferon signaling has reduced anticoagulant activity. In some embodiments, “reduced anticoagulant activity” refers to a heparin polysaccharide having no anticoagulant activity. In alternative embodiments, “reduced anticoagulant activity” refers to a heparin polysaccharide that has less anticoagulant activity than unmodified unfractionated heparin.

Methods of measuring anticoagulant activity are known in the art. For example, reduced anticoagulant activity can be measured using coagulation assays (e.g., which measure clotting times by the heparin under various conditions or measure activated partial thrombloplastin time (APTT)). Some assays to measure reduced coagulation assays determine the coagulation action of the heparin on isolated coagulation enzyme(s) using, for example, specific amidolytic peptide substrates. Non-limiting examples of methods to measure anticoagulant activity are described in Barrowcliffe, T. W., et al. (Journal of pharmaceutical and biomedical analysis 7.2 (1989): 217-226), and Linhardt, Robert J., et al. (Journal of Biological Chemistry 257.13 (1982): 7310-7313), the entire disclosures of which are incorporated herein by reference.

In some embodiments, a heparin polysaccharide that has less anticoagulant activity has 1, 2, 3, 4, 5, 6, 7, 8, 9, 10, 11, 12, 13, 14, 15, 16, 17, 18, 19, 20-fold less anticoagulant activity than the control (e.g., unmodified unfractionated heparin), as measured using a method to determine anticoagulant activity. In some embodiments, a heparin polysaccharide that has less anticoagulant activity has more than 20-fold a reduction in anticoagulant activity than the control heparin polysaccharide (e.g., unmodified unfractionated heparin), as measured using a method to determine anticoagulant activity, such as activated partial thromboplastin time.

In some embodiments, a heparin polysaccharide that has less anticoagulant activity has 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% less anticoagulant activity than the control heparin polysaccharide (e.g., unmodified unfractionated heparin), as measured using a method to determine anticoagulant activity. In some embodiments, the anticoagulant activity, as measured using a method to determine anticoagulant activity, is 20%, 30%, 40%, 50%, 60%, 70%, 80%, or 90% less than that of the control (e.g., unmodified unfractionated heparin).

Without being bound by theory or mechanism, a heparin polysaccharide having reduced anticoagulant activity can be produced by making structural modification to the heparin polysaccharide molecule. There are various methods for producing a heparin polysaccharide of reduced anticoagulant activity, as disclosed herein. In some embodiments, a heparin polysaccharide of reduced anticoagulant activity is a heparin polysaccharide that is desulfated. In some embodiments, a heparin polysaccharide of reduced anticoagulant activity is a heparin polysaccharide that is N-acetylated. Methods for N-acetylating and desulfating molecules are known in the art. In some embodiments, a heparin polysaccharide of reduced anticoagulant activity is a heparin polysaccharide that lacks a unique pentasaccharide sequence (i.e. the antithrombin III binding site) having the following general structure:

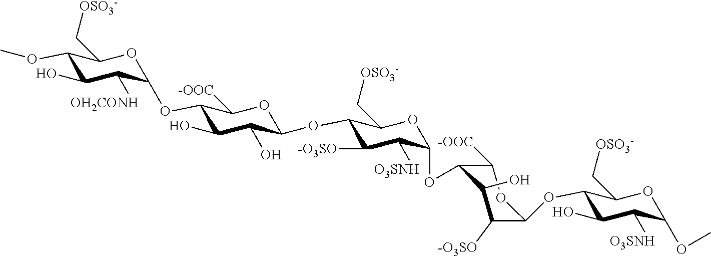

Desulfation can be used to reduce the anticoagulant activity of a heparin polysaccharide. There can be varying degrees of desulfation (i.e. based on the number of desulfation types). Types of desulfation include N-desulfation, and 2-0, 3-0, and 6-0 desulfation. Generally, the anticoagulant activity can be reduced to a greater extent by increasing the degree of O-desulfation (greater number of molecules O-desulfated and/or more types of O-desulfation).

To reduce the anticoagulant activity of a heparin, the AT-bs can be removed. Alternatively, modifications can be made to the AT-bs that affect its binding ability. Examples of modifications that can be made to the AT-bs to reduce or remove anticoagulant activity include, without limitation, 6-O desulfation, 2-O desulfation, N-desulfation, and N-acetylation. 2-O, 3-O desulfated heparin, for example, loses its ability to bind to antithrobmin and factor Xa and has an anticoagulant activity that is about 10-fold lower than undesulfated (and unfractionated) heparin (Rao et al. Am J Physiol Cell Physiol, 2010, 299(1) C97-C110). In some embodiments, a heparin polysaccharide of reduced anticoagulant activity is a heparin polysaccharide with a 6-O-sulfated AT-bs (at the GlcA and/or IdoA2SO3).

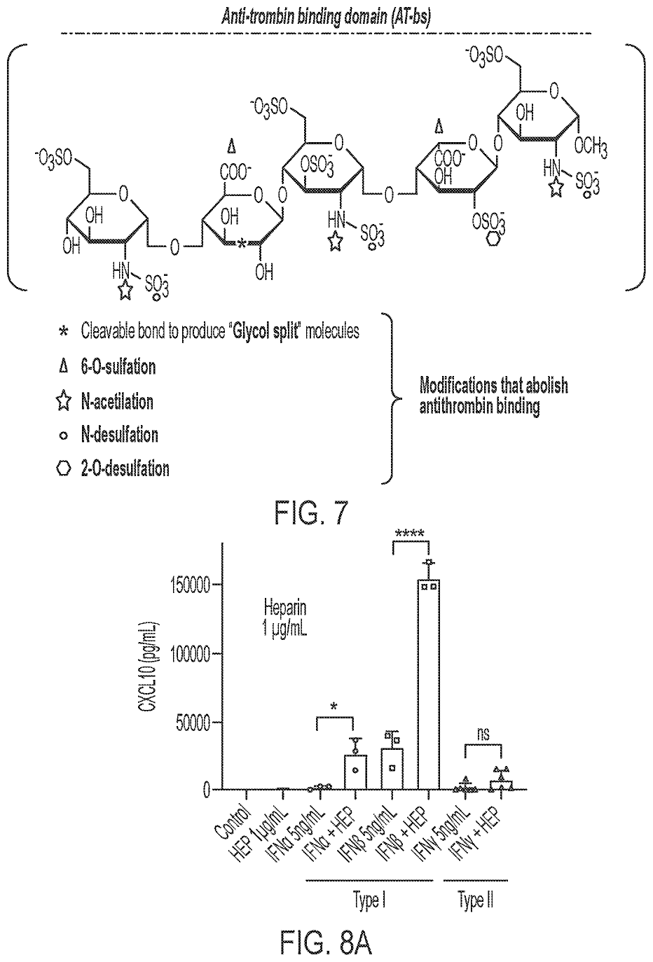

In some embodiments, anticoagulant activity can be reduced or removed by cleavage of the bond between the two hydroxyl groups of the GlcA residue in the AT-bs. This is cleaving or splitting of the C-2-C-3 bonds of nonsulfated uronic acid residues, which can interfere with the biological interactions of heparin by providing flexible joints between protein binding sequences. This process creates a “glycol-split monomer” heparin molecule. An example of a glycol-split monomer is shown in FIG. 7, taken from Poli, Maura, et al. (Blood 123.10 (2014): 1564-1573). A heparin molecule with even less anticoagulant activity can be produced by combining N-acetylation with a glycol-split monomer property.

In some embodiments, a heparin polysaccharide of reduced anticoagulant activity is a heparin polysaccharide that is low molecular weight heparin. In some embodiments of the present invention, the therapeutically effective amount of heparin polysaccharide comprises chains of heparin polysaccharide that are less than 5000 in molecular weight. These chains have reduced anticoagulation activity relative to chains that are longer (e.g., unfractionated heparin). In some embodiments, the average molecular weight of the chains in the therapeutically effective amount of heparin polysaccharide is less than 5000. In some embodiments, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5% or more of the chains in the therapeutically effective amount of heparin polysaccharide are less than 5000 in molecular weight.

### Cancer

The methods and compositions of the present disclosure can be used to treat a subject having cancer. In some embodiments, the cancer is selected from the group consisting of carcinoma, lymphoma, blastoma, sarcoma, and leukemia. In some embodiments, the cancer is breast cancer, for example triple negative breast cancer.

Carcinoma is a cancer that originates in the cells of the skin or tissue lining organs such as the liver or kidneys. Non-limiting examples of types of carcinomas include basal cell carcinoma, squamous cell carcinoma, renal cell carcinoma, ductal carcinoma in situ (DCIS), invasive ductal carcinoma, and adenocarcinoma.

Lymphoma is a cancer that affects the immune system and originates in lymphocytes, which are found throughout the body (e.g., tonsils, lymph nodes, spleen, thymus, bone marrow, etc.). One way to classify lymphomas is to divide them into two categories: Non-Hodgkin's lymphomas and Hodgkin's lymphomas. Non-limiting examples of lymphomas include b-cell lymphoma, t-cell lymphoma, Burkitt's lymphoma, follicular lymphoma, mantle cell lymphoma, primary mediastinal B cell lymphoma, small lymphocytic lymphoma, and Hodgkin's lymphoma (e.g., lymphocyte-depleted Hodgkin's disease, lymphocyte-rich Hodgkin's disease, mixed cellularity Hodgkin's lymphoma, nodular lymphocyte-predominant Hodgkin's disease, nodular sclerosis Hodgkin's lymphoma, etc.).

Blastoma is a type of cancer that is caused by malignancies in precursor cells (e.g., blasts). Blastomas mainly occur in children. Non-limiting examples of blastomas include nephroblastoma, medulloblastoma, retinoblastoma, pulmonary blastoma, hepatoblastoma, medulloblastoma, neuroblastoma, pancreatoblastoma, glioblastoma multiforme, and pleuropulmonary blastoma.

Sarcoma is a general term used for cancers that occur in various locations of the body, mainly originating in the bones and in connective tissue (e.g., fat and muscle). Non-limiting examples of sarcomas include Angiosarcoma, Chondrosarcoma, Dermatofibrosarcoma protuberans, Desmoplastic small round cell tumors, Epithelioid sarcoma, Ewing sarcoma, Gastrointestinal stromal tumor (GIST), Kaposi's sarcoma, Leiomyosarcoma, Liposarcoma, Malignant peripheral nerve sheath tumors, Myxofibrosarcoma, Osteosarcoma, Pleomorphic sarcoma, Rhabdomyosarcoma, Soft tissue sarcoma, Solitary fibrous tumor, Synovial sarcoma, and Undifferentiated pleomorphic sarcoma.

Leukemia is a cancer that originates in the blood-forming tissues (e.g., blood cells, bone marrow, lymphatic system) and bone marrow. Rather than forming a tumor, leukemias are known to cause excess abnormal white blood cells. Non-limiting examples of types of leukemia include acute lymphocytic leukemia (ALL), chronic lymphocytic leukemia (CLL), acute myelogenous leukemia (AML), and chronic myelogenous leukemia (CML).

In some embodiments of the present disclosure, the cancer treated using the disclosed methods and compositions is lung cancer or glioblastoma. In some embodiments of the present disclosure, the cancer treated using the disclosed methods and compositions is a small cell lung cancer (SCLC). In some embodiments of the present disclosure, the cancer treated using the disclosed methods and compositions is non-small cell lung cancer (NSCLC). In some embodiments of the present disclosure, the cancer treated using the disclosed methods and compositions is a mesothelioma. In some embodiments of the present disclosure, the cancer treated using the disclosed methods and compositions is a meningioma.

Small cell lung cancer (SCLC) is an aggressive form of lung cancer that usually originated in the bronchi. Non-limiting examples of SCLCs that are contemplated herein include small cell carcinoma (also referred to as oat cell cancer) and combined small cell carcinoma.

Mesothelioma is an aggressive cancer that affects the lining of the lungs, heart, or abdomen. Mesotheliomas can be classified based on the location in the body where the tumors originate. Non-limiting examples of types of mesotheliomas that are contemplated herein include pleural mesothelioma, peritoneal mesothelioma, pericardial mesothelioma, and testicular mesothelioma. Mesotheliomas can also be classified by the cell type of the tumor. Non-limiting examples of types of mesotheliomas (based on cell type) that are contemplated herein include epithelioid, biphasic and sarcomatoid mesotheliomas.

Meningioma is a tumor that forms on the meninges—the membranes covering the brain and spinal cord. All cancers classified as meningiomas are contemplated herein. Non-limiting examples of types of meningiomas that are contemplated include clival meningioma, convexity meningioma, foramen magnum meningioma, olfactory groove meningioma, posterior fossa meningioma, suprasellar meningioma, falcine and parasagittal meningiomas, intraventricular meningiomas, cavernous sinus meningiomas, sphenoid wing meningiomas, spinal meningiomas and tentorial meningiomas.

Breast cancer is cancer that forms in the cells of the breast. In some cases it originates in the milk-producing ducts (e.g., invasive ductal carcinoma). Breast cancer may also begin in the glandular tissue called lobules (e.g., invasive lobular carcinoma) or in other cells or tissue within the breast. Non-limiting examples of breast cancers include angiosarcoma, ductal carcinoma in situ (DCIS), inflammatory breast cancer, invasive lobular carcinoma, lobular carcinoma in situ (LCIS), male breast cancer, Paget's disease of the breast, and recurrent breast cancer. In some embodiments of the present invention, the breast cancer is a metastatic breast cancer. In some embodiments, the breast cancer is at stage I, stage II, or stage III. In some embodiments, the breast cancer is deficient in homologous recombination DNA repair. In some embodiments, the breast cancer has impaired function of BRCA1 or BRCA2. In some embodiments, the breast cancer is negative for at least one of: estrogen (ER), progesterone (PR), or human epidermal growth factor receptor 2 (HER2), optionally wherein the breast cancer is positive for at least one of ER, PR or HER2. In some embodiments, the breast cancer is triple negative breast cancer.

Triple Negative Breast Cancer is a form of breast cancer in which the three most common types of receptors associated with most breast cancer growth-estrogen, progesterone, and the HER-2/neu gene—are not present in the cancer tumor. This type of breast cancer is particularly challenging to treat because it does not respond to hormonal therapy medications that target these receptors.

In some embodiments, the cancer that can be treated using methods or compositions of the present disclosure is selected from the group consisting of cancers of the lung, bone, pancreas, skin, head, neck, uterus, ovaries, stomach, colon, breast, esophagus, small intestine, bowel, endocrine system, thyroid gland, parathyroid gland, adrenal gland, urethra, prostate, penis, testes, ureter, bladder, kidney or liver; rectal cancer, cancer of the anal region, carcinomas of the fallopian tubes, endometrium, cervix, vagina, vulva, renal pelvis, renal cell, sarcoma of soft tissue, myxoma, rhabdomyoma, fibroma, lipoma, teratoma, cholangiocarcinoma, hepatoblastoma, angiosarcoma, hemangioma, hepatoma, fibrosarcoma, chondrosarcoma, myeloma, chronic or acute leukemia, lymphocytic lymphomas, primary CNS lymphoma, neoplasms of the CNS, spinal axis tumors, squamous cell carcinomas, synovial sarcoma, malignant pleural mesotheliomas, brain stem glioma, pituitary adenoma, meningioma, bronchial adenoma, chondromatous hanlartoma, inesothelioma, Hodgkin's Disease, brain (gliomas), glioblastomas, astrocytomas, glioblastoma multiforme, Bannayan-Zonana syndrome, Cowden disease, Lhermitte-Duclos disease, Wilm's tumor, Ewing's sarcoma, Rhabdomyosarcoma, ependymoma, medulloblastoma, melanoma, ovarian, pancreatic, adenocarcinoma, ductal madenocarcinoma, adenosquamous carcinoma, small cell lung cancer, non-small cell lung cancer, acinar cell carcinoma, glucagonoma, insulinoma, prostate, sarcoma, osteosarcoma, giant cell tumor of bone, thyroid, lymphoblastic T cell leukemia, chronic myelogenous leukemia, chronic lymphocytic leukemia, hairy-cell leukemia, acute lymphoblastic leukemia, acute myelogenous leukemia, chronic neutrophilic leukemia, acute lymphoblastic T cell leukemia, plasmacytoma, Immunoblastic large cell leukemia, mantle cell leukemia, multiple myeloma, megakaryoblastic leukemia, multiple myeloma, acute megakaryocyte leukemia, pro myelocytic leukemia, erythroleukemia, malignant lymphoma, hodgkins lymphoma, non-hodgkins lymphoma, lymphoblastic T cell lymphoma, Burkitt's lymphoma, follicular lymphoma, neuroblastoma, bladder cancer, urothelial cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor) and testicular cancer.

Also contemplated in the present disclosure are methods for treating cells in vitro, comprising administering a STING agonist and heparin polysaccharide to one or more cells or tissue. The cells can be cancerous or non-cancerous. In some embodiments, the cells are treated with the STING agonist and heparin polysaccharide composition to determine response to the treatment or effectiveness of the treatment. Non-limiting examples of cells that are contemplated include SCLC cells, mesothelioma cells, and meningioma cells. SCLC cell types include, without limitation, H69M cells. Mesothelioma cell types include, without limitation, MS428, H2052, and MS924 cell types. Meningioma cell types include, without limitation HBL52 and Ben-Men-1 cell types.

### Chemotherapeutic Agents

In some embodiments of the present disclosure, the heparin polysaccharide and the stimulator of interferon signaling are administered with an additional chemotherapeutic (also referred to as “anticancer”) agent, optionally a checkpoint inhibitor. The term “chemotherapeutic agent” refers to a therapeutic agent known to be of use in the treatment of cancer.

An anticancer agent can be, without limitation, a protein, a nucleic acid, a small molecule, or a drug for the treatment of cancer. This anticancer agent can have any anti-cancer effect on the population of cells that it is administered to including, but not limited to, a cytotoxic, apoptotic, anti-mitotic anti-angiogenesis or inhibition of metastasis effect. This anticancer agent can also affect DNA damage response (e.g., a DNA repair inhibitor). In some embodiments, the additional anticancer agent is a drug directed against overexpressed protein products.

Anticancer agents include, without limitation, antimetabolites, inhibitors of topoisomerase I and II, alkylating agents and microtubule inhibitors (e.g., taxol). Non-limiting examples of anticancer agents include adriamycin aldesleukin; alemtuzumab; alitretinoin; allopurinol; altretamine; amifostine; anastrozole; arsenic trioxide; Asparaginase; BCG Live; bexarotene capsules; bexarotene gel; bleomycin; busulfan intravenous; busulfan oral; calusterone; capecitabine; carboplatin; carmustine; carmustine with Polifeprosan 20 Implant; celecoxib; chlorambucil; cisplatin; cladribine; cyclophosphamide; cytarabine; cytarabine liposomal; dacarbazine; dactinomycin; actinomycin D; Darbepoetin alfa; daunorubicin liposomal; daunorubicin, daunomycin; Denileukin diftitox, dexrazoxane; docetaxel; doxorubicin; doxorubicin liposomal; Dromostanolone propionate; Elliott's B Solution; epirubicin; Epoetin alfa estramustine; etoposide phosphate; etoposide (VP-16); exemestane; Filgrastim; floxuridine (intraarterial); fludarabine; fluorouracil (5-FU); fulvestrant; gemcitabine, gemtuzumab ozogamicin; goserelin acetate; hydroxyurea; Ibritumomab Tiuxetan; idarubicin; ifosfamide; imatinib mesylate; Interferon alfa-2a; Interferon alfa-2b; irinotecan; letrozole; leucovorin; levamisole; lomustine (CCNU); meclorethamine (nitrogen mustard); megestrol acetate; melphalan (L-PAM); mercaptopurine (6-MP); mesna; methotrexate; methoxsalen; mitomycin C; mitotane; mitoxantrone; nandrolone phenpropionate; Nofetumomab; LOddC; Oprelvekin; oxaliplatin; paclitaxel; pamidronate; pegademase; Pegaspargase; Pegfilgrastim; pentostatin; pipobroman; plicamycin; mithramycin; porfimer sodium; procarbazine; quinacrine; Rasburicase; Rituximab; Sargramostim; streptozocin; talbuvidine (LDT); talc; tamoxifen; temozolomide; teniposide (VM-26); testolactone; thioguanine (6-TG); thiotepa; topotecan; toremifene; Tositumomab; Trastuzumab; tretinoin (ATRA); uracil mustard; valrubicin; valtorcitabine (monoval LDC); vinblastine; vinorelbine; zoledronate; and mixtures thereof, among others (see U.S. Pat. No. 9,643,922, the relevant disclosures of which are herein incorporated by reference).

Non-limiting examples of anticancer agents include oestrogen receptor modulators, androgen receptor modulators, retinoid receptor modulators, cytotoxic agents, antiproliferative agents, prenyl-protein transferase inhibitors, HMG-CoA reductase inhibitors, reverse transcriptase inhibitors, poly ADP ribose polymerase (PARP) inhibitors, aurora kinase inhibitors, and further angiogenesis inhibitors.

Non-limiting examples of retinoid receptor modulators include bexarotene, tretinoin, 13-cis-retinoic acid, 9-cis-retinoic acid, .alpha.-difluoromethylornithine, ILX23-7553, trans-N-(4′-hydroxyphenyl)retinamide and N-4-carboxyphenylretinamide (see U.S. Pat. No. 10,093,623, the relevant disclosures of which are herein incorporated by reference).

Non-limiting examples of cytotoxic agents include tirapazimine, sertenef, cachectin, ifosfamide, tasonermin, lonidamine, carboplatin, altretamine, prednimustine, dibromodulcitol, ranimustine, fotemustine, nedaplatin, oxaliplatin, temozolomide, heptaplatin, estramustine, improsulfan tosylate, trofosfamide, nimustine, dibrospidium chloride, pumitepa, lobaplatin, satraplatin, profiromycin, cisplatin, irofulven, dexifosfamide, cis-aminedichloro(2-methylpyridine)platinum, benzylguanine, glufosfamide, GPX100, (trans,trans,trans)bis-mu-(hexane-1,6-diamine)-mu-[diamineplatinum(II)]bi-s[diamine(chloro)platinum(II)]tetrachloride, diarisidinylspermine, arsenic trioxide, 1-(11-dodecylamino-10-hydroxyundecyl)-3,7-dimethylxanthine, zorubicin, idarubicin, daunorubicin, bisantrene, mitoxantrone, pirarubicin, pinafide, valrubicin, amrubicin, antineoplaston, 3′-deamino-3′-morpholino-13-deoxo-10-hydroxycarminomycin, annamycin, galarubicin, elinafide, MEN10755 and 4-demethoxy-3-deamino-3-aziridinyl-4-methylsulfonyldaunorubicin (see WO 00/50032, the relevant disclosures of which are herein incorporated by reference).

Non-limiting examples of antiproliferative agents include antisense RNA and DNA oligonucleotides such as G3139, ODN698, RVASKRAS, GEM231 and INX3001 and antimetabolites such as enocitabine, carmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, nelzarabine, 2′-deoxy-2′-methylidenecytidine, 2′-fluoromethylene-2′-deoxycytidine, N-[5-(2,3-dihydrobenzofuryl)sulfonyl]-N′-(3,4-dichlorophenyl)urea, N6-[4-deoxy-4-[N2-[2(E),4(E)-tetradecadienoyl]-glycylamino]-L-glycero-B-L--mannoheptopyranosyl]adenine, aplidine, ecteinascidin, troxacitabine, 4-[2-amino-4-oxo-4,6,7,8-tetrahydro-3H-pyrimidino[5,4-b]-1,4-thiazin-6-yl-(S)ethyl]-2,5-thienoyl-L-glutamic acid, aminopterin, 5-fluorouracil, alanosine, 11-acetyl-8-(carbamoyloxymethyl)-4-formyl-6-methoxy-14-oxa-1,11-diazatetr-acyclo(7.4.1.0.0)tetradeca-2,4,6-trien-9-ylacetic acid ester, swainsonine, lometrexol, dexrazoxane, methioninase, 2′-cyano-2′-deoxy-N4-palmitoyl-1-B-D-arabinofuranosyl cytosine and 3-aminopyridine-2-carboxaldehyde thiosemicarbazone. “Antiproliferative agents” also include monoclonal antibodies to growth factors other than those listed under “angiogenesis inhibitors”, such as trastuzumab (for examples, see U.S. Pat. No. 6,069,134, the relevant disclosures of which are herein incorporated by reference).

Non-limiting examples of poly ADP ribose polymerase (PARP) inhibitors include Olaparib, Rucaparib, Niraparib, Talazoparib, Veliparib, BGB-290 (Pamiparib), CEP 9722, E7016, Iniparib (BSI 201), and 3-aminobenzamide. Examples of PARP inhibitors are known in the art and are described, for example, in CR Calebrese, et al., Clin. Cancer Res., Vol. 9, 2711-18 (2003), Veuger S J, et al., Cancer Res. Vol. 63.6008 to 15 (2003); C R Calabrese et al., J. Nat'l. Cancer Inst 96 (1), 56-67 (2004); “Potent Novel PARP Inhibitors,” Expert Reviews in Molecular Medicine, vol. 7 (4) (March 2005); and P. Jagtap, Nature Rev.: Drug Discovery, vol. 4: 421-40 (2005), the relevant disclosures of which are herein incorporated by reference. benzamides, quinolones and isoquinolones, benzopyrones, methyl 3,5-diiodo-4-(4′-methoxyphenoxy) benzoate, and methyl-3,5-diiodo-4-(4′-methoxy-3′,5′-diiodo-phenoxy) benzoate (U.S. Pat. Nos. 5,464,871, 5,670,518, 6,004,978, 6,169,104, 5,922,775, 6,017,958, 5,736,576, and 5,484,951, the relevant disclosures of which are herein incorporated by reference). The PARP inhibitors include a variety of cyclic benzamide analogs (i.e. lactams) which are potent inhibitors at the NAD site. Other PARP inhibitors include, but are not limited to, benzimidazoles and indoles (see, for example, EP841924, EP1127052, U.S. Pat. Nos. 6,100,283, 6,310,082, US2002/156050, US2005/054631, WO05/012305, WO99/11628, and US2002/028815, the relevant disclosures of which are herein incorporated by reference).

Non-limiting examples of aurora kinase inhibitors include Examples of aurora kinase inhibitors include, but are not limited Binucleine 2, which is also known as Methanimidamide, N′-[1-(3-chloro-4-fluorophenyl)-4-cyano-5-yl batch-1H-]-N, N-dimethyl. Non-limiting examples of aurora kinase inhibitors include the compounds disclosed in, for example, WO 05/111039, US2005/0256102, US2007/0185087, WO 08/021038, US2008/0045501, WO 08/063525, US2008/0167292, WO 07/113212, EP1644376, US2005/0032839, WO 05/005427, WO 06/070192, WO 06/070198, WO 06/070202, WO 06/070195, WO 06/003440, WO 05/002576, WO 05/002552, WO 04/071507, WO 04/058781, WO 06/055528, WO 06/055561, WO 05/118544, WO 05/013996, WO 06/036266, US2006/0160874, US2007/0142368, WO 04/043953, WO 07/132220, WO 07/132221, WO 07/132228, WO 04/00833 and WO 07/056164, the relevant disclosures of which are herein incorporated by reference.

### Immune Checkpoint Inhibitors

In some embodiments of the present disclosure, the heparin polysaccharide (optionally desulfated) and stimulator of interferon signaling are administered with a checkpoint inhibitor (e.g., a PD-1 inhibitor or PD-L1 inhibitor) to treat a subject having cancer. In some embodiments, the heparin polysaccharide (e.g., desulfated), stimulator of interferon signaling, and a checkpoint inhibitor is combined to treat a subject having any of the cancers contemplated herein. Lung cancer and glioblastoma are of particular interest for such treatments based on the clinical need to enhance checkpoint therapy response and immunohistochemistry demonstrating STING expression in the absence of activation (absent phospo-TBK1).

In some embodiments, the heparin polysaccharide (optionally desulfated) and stimulator of interferon signaling are administered with a PD-L1 inhibitor to treat a subject having cancer, and the PD-L1 inhibitor is atezolizumab (MPDL3280A), optionally wherein the cancer is SCLC.

Checkpoint Inhibitors (also referred to as immune checkpoint inhibitors) are drugs or drug candidates that inhibit/block the inhibitory checkpoint proteins. Checkpoint proteins help keep immune responses in check and prevent the immune system from targeting cells indiscriminately. There are stimulatory checkpoint proteins that promote an immune response (e.g., T-cell proliferation) and inhibitory checkpoint proteins that protect cells from an immune response. Inhibitory checkpoint proteins can facilitate tumor-cell survival. Non-limiting examples of inhibitory checkpoint proteins include programmed death-1 (PD-1), programmed death-ligand 1 (PD-L1), adenosine A2A receptor (A2AR), Cluster of Differentiation 276 (CD276), V-Set Domain Containing T Cell Activation Inhibitor 1 (VTCN1), B- and T-lymphocyte attenuator (BTLA), Indoleamine-pyrrole 2,3-dioxygenase (IDO), Killer-cell Immunoglobulin-like Receptor (KIR), Lymphocyte Activation Gene-3 (LAG3), NADPH oxidase 2 (NOX2), T-cell Immunoglobulin domain and Mucin domain 3 (TIM-3), V-domain Ig suppressor of T cell activation (VISTA) protein, Sialic acid-binding immunoglobulin-type lectin 7 (SIGLEC7), and cytotoxic T lymphocyte antigen-4 (CTLA-4).

PD-L1 is expressed on tumor cells and PD-1 is expressed on T cells. The binding of PD-L1 to PD-1 prevents T cells from killing tumor cells in the body. Blocking the binding of PD-L1 to PD-1 with an immune checkpoint inhibitor using an inhibitor that specifically binds to PD-L1 or PD-1 (also referred to an antagonists of PD-1 or an antagonist of PD-L1, e.g., anti-PD-L1 or anti-PD-1) allows the T cells to kill tumor cells. There is evidence in the literature that immune check point inhibition therapy can be enhanced by stimulating an increase in expression of inhibitory check point proteins.

Non-limiting examples of checkpoint inhibitors contemplated for use in the present invention include anti-CTLA-4 molecules, anti-PD1 molecules, and anti-PD-L1 molecules. Non-limiting examples of checkpoint inhibitors contemplated for use in the present invention include: Tremelimumab (CP-675,206), a human IgG2 monoclonal antibody with high affinity to CTLA-4; Ipilimumab (MDX-010), a human IgG1 monoclonal antibody to CTLA-4; Nivolumab (BMS-936558), a human monoclonal anti-PD1 IgG4 antibody that essentially lacks detectable antibody-dependent cellular cytotoxicity (ADCC); MK-3475 (Pembrolizumab; formerly lambrolizumab), a humanized IgG4 anti-PD-1 antibody that contains a mutation at C228P designed to prevent Fc-mediated ADCC; Urelumab (BMS-663513), a fully human IgG4 monoclonal anti-CD137 antibody; anti-LAG-3 monoclonal antibody (BMS-986016); Atezolizumab (MPDL3280A), and anti-PD-L1 antibody; Avelumab (MSB0010718C), an anti-PD-L1 antibody; Durvalumab (MEDI4736), an anti-PD-L1 antibody; Cemiplimab (REGN-2810), an anti-PD1 antibody; and Bavituximab (chimeric 3G4), a chimeric IgG3 antibody against phosphatidylserine.

A PD-1 inhibitor, as used herein, is an agent that inhibits or prevents PD-1 activity. The activity can be reduced in a cell or a subject, for example, by 5%, 10%, 20%, 30%, 40%, 50%, 60%, 70%, 80%, 90%, 100% or more, compared a cell or subject that has not been exposed to the PD-1 inhibitor. In some embodiments, a PD-1 inhibitor is an antibody that specifically binds to PD-1 to inhibit or prevent PD-1 activity. In some embodiments, a PD-1 inhibitor is an agent that inhibits the expression of DNA or mRNA encoding PD-1 (e.g., inhibitory nucleic acids). A PD-1 inhibitor can include proteins (such as fusion proteins), small molecules, and peptides, e.g., peptide mimetics of PD-L1 and PD-L2 that bind PD-1 but do not activate PD-1.

Non-limiting examples of PD-1 inhibitors include nivolumab (e.g., OPDIVO® from Bristol-Myers Squibb); pidilizumab (e.g., CT-011 from CureTech); MK-3475 (Merck) 1; pembrolizumab (e.g., KEYTRUDA® from Merck); MEDI-0680 (AstraZeneca/MedImmune); AMP-224 (Glaxo Smith Kline and Amplimmune); and REGN2810 (Regeneron/Sanofi). Non-limiting examples of PD-1 inhibitor are described in U.S. Publication Numbers 20130280265, 20130237580, 20130230514, 20130109843, 20130108651, 20130017199, 20120251537, and 20110271358, and in European Patent EP2170959B1, the entire disclosures of which are incorporated herein by reference. Additional examples of PD-1 inhibitors are described in Curran et al., PNAS, 107, 4275 (2010); Topalian et al., New Engl. J. Med. 366, 2443 (2012); Brahmer et al., New Engl. J. Med. 366, 2455 (2012); Dolan et al., Cancer Control 21, 3 (2014); and Sunshine et al., Curr. Opin. in Pharmacol. 23 (2015), the entire disclosures of which are incorporated herein by reference.

A PD-L1 inhibitor, as used herein, is an agent that inhibits or prevents PD-L1 activity. The activity can be reduced in a cell or a subject, for example, by 5%, 10%, 20%, 30%, 40%, 50%, 60%, 70%, 80%, 90%, 100% or more, compared a cell or subject that has not been exposed to the PD-L1 inhibitor. In some embodiments, a PD-L1 inhibitor is an antibody that specifically binds to PD-L1 to inhibit or prevent PD-L1 activity. In some embodiments, a PD-L1 inhibitor is an agent that inhibits the expression of DNA or mRNA encoding PD-L1 (e.g., inhibitory nucleic acids). A PD-L1 inhibitor can include proteins (such as fusion proteins), small molecules, and peptides, e.g., peptide mimetics of PD-1 that bind PD-L1 but do not activate PD-L1.

Non-limiting examples of PD-L1 inhibitors include atezolizumab (also called MPDL3280A or TECENTRIQ™, Genentech/Roche); MEDI4736 (AstraZeneca/MedImmune); BMS-936559 (Bristol-Meyers Squibb); avelumab (also called MSB 0010718C Merck KGaA/Pfizer); and CA-170 (Aurigene/Curis). Non-limiting examples of PD-L1 inhibitors are described in U.S. Publication Numbers 20090055944, 20100203056, 20120039906, 20130045202, 20130309250, and 20160108123, the entire disclosures of which are incorporated herein by reference.

### Antithrombotic Therapy and Thrombolytic Therapy

In some embodiments of the present invention, a therapeutically effective amount of a stimulator of interferon signaling and a therapeutically effective amount of heparin polysaccharide are administered to a subject that is not receiving concurrent antithrombotic therapy or thrombolytic therapy. For example, the therapeutically effective amount of a stimulator of interferon signaling and therapeutically effective amount of heparin polysaccharide are administered to a subject that is not a candidate for antithrombotic therapy or thrombolytic therapy. An example of such a subject is one that is unlikely to have received heparin (e.g., due to a contraindication for heparin). Such a subject may be unlikely to have received heparin and chemotherapy. In some embodiments, such a subject is unlikely to have (or did not) received heparin in the past. In some cases, the subject may be unlikely to have received heparin within the last 45 minutes, within the last 60 minutes, within the last 90 minutes or within the last 120 minutes or more. In some cases, the subject may be unlikely to have received heparin within the time required to clear a dose (e.g., large dose, e.g., 28,000 units) from the subject's body.

“Antithrombotic therapy” refers to treatment of a subject with antithrombotic drugs. Antithrombotic drugs function to prevent or retard clot formation. A clot or “thrombus” is comprised of fibrin and platelets. They facilitate wound healing; however, their formation in a blood vessel can be detrimental (and sometimes fatal). Some antithrombotic drugs slow down (or prevent) fibrin formation and the consequent clotting, in which case they are classified as anticoagulant drugs. Other antithrombotic drugs prevent platelet clumping and the consequent clot formation—these are classified as antiplatelet drugs.

Heparin is an anticoagulant that is typically administered intravenously and typically acts immediately on subjects. Non-limiting examples of anticoagulants and antithrombotic agents include: warfarin, dalteparine, heparine, tinzaparin, enoxaparin, danaparoid, abciximab, alprostadil, altiplase, anagralide, anistreplase, argatroban, ataprost, betaprost, camonagrel, cilostazol, clinprost, clopidogrel, cloricromen, dermatan, desirudine, domitroban, drotaverine, epoprostenol, eptifibatide, fradafiban, gabexate, iloprost, isbogrel, lamifiban, lamoteplase, lefradafiban, lepirudin, levosimendan, lexipafant, melagatran, nafagrel, nafamostsat, nizofenone, orbifiban, ozagrel, pamicogrel, parnaparin, quinobendan, reteplase, sarpogralate, satigrel, silteplase, simendan, ticlopidine, vapiprost, tirofiban, xemilofiban, Y20811, and salts thereof, esters thereof, hydrates thereof, polymorphs thereof and isomers thereof.

Non-limiting examples of antiplatelet drugs include nonsteroidal antiinflammatory drugs (NSAIDS) such as acetaminophen, aspirin, codeine, diclofenac, droxicam, fentanyl, ibuprofen, indomethacin, ketorolac, mefenamate, morphine, naproxen, phenacetin, piroxicam, sufentanil salts, sulfinpyrazone, sulindac, and pharmaceutically acceptable salts thereof. NSAIDs, aspirin (acetylsalicylic acid or ASA) and piroxicam are preferred. Other inhibitors suitable platelet include blockers glycoprotein IIb/IIIa (e.g., abciximab, eptifibatide, tirofiban, Integrelin) receptor antagonists thromboxane A2 (e.g., ifetroban), inhibitors of thromboxane-A2-synthetase inhibitors, phosphodiesterase III (PDE-III) (e.g., dipyridamole, cilostazol), and phosphodiesterase type 5 (PDE V) (e.g., sildenafil), antagonists activated receptor 1 protease (PAR-1) (for example, SCH-530348, SCH-203099, SCH-529153, and SCH205 831), and their pharmaceutically acceptable salts.

In some embodiments, a subject that is not receiving concurrent antithrombotic therapy (e.g., not a candidate for antithrombotic therapy) can be a subject having a contraindication (absolute or relative) for antithrombotic therapy. Non-limiting examples of contraindications for antithrombotic therapy include bleeding abnormality (e.g., thrombocytopenia, platelet defect, peptic ulcer disease), central nervous system (CNS) lesion (e.g., stroke, surgery, trauma), spinal anesthesia, lumbar puncture, malignant hypertension, advanced retinopathy, renal insufficiency, active gastrointestinal bleed, known large esophageal varices, significant thrombocytopenia (e.g., platelet count <50×10⁹/L), recent (e.g., within 72 hours) major surgery with risk of severe bleeding, previously documented or known hypersensitivity to antithrombotic drugs, active bleeding or bleeding risk (e.g., within 3 months), decompensated liver disease, deranged baseline clotting screen (international normalized ratio (INR)>1.5), pregnancy, recent pregnancy (e.g., within 48 hours post-partum), severe renal impairment (e.g., Glomerular Filtration Rate (GFR)<30 mL/min/1.73 m² or on dialysis). In some embodiments, non-limiting examples of contraindications for antithrombotic therapy include previous history intracranial hemorrhage, recent (e.g., within 6 months) major extracranial bleed, recent (e.g., within 3 months) peptic ulcer (PU); age >65 years; previous history bleed or predisposition to bleeding (e.g., diverticulitis); uncontrolled hypertension; severe renal impairment (e.g., serum creatinine >200 umol/L, GFR<30 mL/min/1.73 m² or on dialysis), acute hepatic impairment (e.g., bilirubin >2×ULN (upper limit of the normal range)+LFTs (liver function tests) >3×ULN), chronic liver disease (e.g. cirrhosis), low platelet count <80×10⁹/L, thrombocytopenia, anemia of undiagnosed cause, and patient on concomitant drugs associated with an increased bleeding risk (e.g., SSRIs, oral steroids, NSAIDs, methotrexate or other immune-suppressant agents).

Thrombolytic therapy (also referred to as thrombolysis or fibrinolytic therapy) is the treatment of a subject with drugs that target and dissolve (lyse) blood clots formed in blood vessels. Thrombolytic therapy can help restore blood flow to an organ or body part when the clot has led to an occlusion of a blood vessel. Due to the serious effects of occluded blood vessels (particularly in cases of occlusion of major blood vessels) thrombolytic therapy is time sensitive and more effective when initiated early.

Thrombolytic therapy is usually administered intravenously and it is often administered in combination with heparin. Examples of disorders that thrombolytic therapy is used to treat included ST elevation myocardial infarction, stroke, massive pulmonary embolism, deep vein thrombosis, acute limb ischemia, and clotted hemothorax. Thrombolytic therapy is used for emergency treatment for strokes and heart attacks.

Non-limiting examples of drugs for thrombolytic therapy include tissue plasminogen activator—t-PA—alteplase (Activase), recombinant tissue plasminogen activators (rtPA), reteplase (Retavase), tenecteplase (TNKase), anistreplase (Eminase), streptokinase (Kabikinase, Streptase) and urokinase (Abbokinase). Additional examples of thrombolytic drugs can be found in various well known reference works (e.g., Budavari et al. The Merck index. Vol. 11. Rahway, N.J.: Merck, 1989).

In some embodiments, a subject that is not receiving concurrent thrombolytic therapy (e.g., not a candidate for thrombolytic therapy) can be a subject having a contraindication (absolute or relative) for thrombolytic therapy. Non-limiting examples of contraindications for thrombolytic therapy include any previous history of hemorrhagic stroke; ischemic stroke within 3 months; any prior intracranial hemorrhage; a history of stroke, dementia, or central nervous system damage within 1 year; head trauma or facial trauma within 3 weeks; brain surgery within 6 months; known intracranial neoplasm; known structural cerebral vascular lesion; suspected aortic dissection; internal bleeding within 6 weeks; active bleeding (excluding menses) within 3 hours or more; intracranial or intraspinal surgery within 2 months; known bleeding disorder; traumatic cardiopulmonary resuscitation within 3 weeks; advanced liver disease; uncontrolled hypertension (e.g., systolic blood pressure >180 mm Hg, diastolic blood pressure >110 mm Hg); puncture of noncompressible blood vessel within 2 weeks; major surgery, trauma, or bleeding within 2 weeks; coma or severe obtundation with fixed eye deviation and complete hemiplegia; septic embolus; elevated Activated Prothrombin Time (APTT); known hereditary or acquired haemorrhagic diathesis; International normalized ration (INR) >1.5; INR >1.7; advanced right heart failure; anticoagulation; platelet count <100,000 uL; and serum glucose <2.8 mmol/l or >22.0 mmol/l. In some embodiments, non-limiting examples of contraindications for thrombolytic therapy include severe neurological impairment with NIH stroke scale/score (NIHSS) score >22; age >80 years; age >75 years; CT evidence of extensive middle cerebral artery (MCA) territory infarction (sulcal effacement or blurring of grey-white junction in greater than ⅓ of MCA territory); stroke or serious head trauma within the past 3 months where the risks of bleeding are considered to outweigh the benefits of therapy; major surgery within the last 14 days; known history of intracranial hemorrhage, subarachnoid hemorrhage, known intracranial arteriovenous malformation or previously known intracranial neoplasm; suspected recent (e.g., within 30 days) myocardial infarction; cardiopulmonary resuscitation >10 minutes; recent (e.g., 2-4 weeks) internal bleeding; major surgery, e.g., within 3 weeks; recent (e.g., within 30 days) biopsy of a parenchymal organ or surgery that, in the opinion of the responsible clinician, would increase the risk of unmanageable (e.g., uncontrolled by local pressure) bleeding; recent (e.g., within 30 days) trauma with internal injuries or ulcerative wounds; active peptic ulcer; gastrointestinal or urinary tract hemorrhage, e.g., within the last 30 days; any active or recent hemorrhage that, in the opinion of the responsible clinician, would increase the risk of unmanageable (e.g., by local pressure) bleeding; arterial puncture at non-compressible site, e.g., within the last 7 days; concomitant serious, advanced or terminal illness or any other condition that, in the opinion of the responsible clinician would increase the risk of unmanageable bleeding; seizure; and pregnancy.

In some embodiments, a subject that is not receiving concurrent antithrombotic therapy or thrombolytic therapy (e.g., not a candidate for antithrombotic therapy or thrombolytic therapy) can be a subject having a cancer such as, but not limited to, meningioma, glioma, medulloblastoma, pituitary adenomas, primary CNS lymphomas. In some embodiments, a subject that is not receiving concurrent antithrombotic therapy or thrombolytic therapy (e.g., not a candidate for antithrombotic therapy or thrombolytic therapy) can be a subject having a cancer associated with CNS germ cell tumors (e.g., germinomatous germ cell tumors or non-germinomatous germ cell tumors). There are several sub-types of non-germinomatous germ cell tumor, including (without limitation) teratomas, choriocarcinomas, endodermal sinus tumors (yolk sac tumors), embryonal carcinomas and mixed tumors. In some embodiments, a subject that is not receiving concurrent antithrombotic therapy or thrombolytic therapy (e.g., not a candidate for antithrombotic therapy or thrombolytic therapy) can be a subject that is prone to, recently had (e.g., within 24 hours, 2 days, 4 days, 1 week, 3 weeks, 1 month, 2 months, 3 months, or more) or is currently having intracranial bleeding. In some embodiments, a subject that is not receiving concurrent antithrombotic therapy or thrombolytic therapy (e.g., not a candidate for antithrombotic therapy or thrombolytic therapy) can be a subject that is undergoing brain surgery or surgery on the central nervous system (CNS). In some embodiments, a subject that is not receiving concurrent antithrombotic therapy or thrombolytic therapy (e.g., not a candidate for antithrombotic therapy or thrombolytic therapy) can be a subject that has or is at risk of having hepatic damage (or hepatic failure), has a history of hepatic failure or is currently experiencing hepatic failure (e.g., chronic hepatic failure).

In some of the foregoing embodiments, the subject will be treated with a heparin polysaccharide of reduced anticoagulation activity.

### Pharmaceutical Compositions

In some aspects, the present disclosure provides pharmaceutical compositions comprising a stimulator of interferon signaling, heparin polysaccharide, and a pharmaceutically acceptable excipient. These pharmaceutical compositions may comprise one or more organic solvents.

In certain embodiments, the pharmaceutical compositions do not include organic solvent. In certain embodiments, organic solvents are not used in the preparation of the compositions. In certain embodiments, the pharmaceutical compositions are free of organic solvent. In certain embodiments, the pharmaceutical compositions are substantially free of organic solvent. In certain embodiments, the pharmaceutical compositions comprise, by weight, less than 10%, less than 5%, less than 4%, less than 3%, less than 2%, less than 1%, less than 0.5%, less than 0.1%, less than 0.01%, less than 0.001%, or less than 0.0001% of organic solvent. In certain embodiments, the pharmaceutical compositions comprise, by weight, less than 1000 ppm, less than 500 ppm, less than 400 ppm, less than 300 ppm, less than 200 ppm, less than 100 ppm, less than 50 ppm, less than 40 ppm, less than 30 ppm, less than 20 ppm, less than 10 ppm, less than 1 ppm, less than 10 ppb, or less than 1 ppb of organic solvent.

In certain embodiments, the pharmaceutical compositions comprise organic solvent. In certain embodiments, the organic solvent is cyclodextrin, methanol, ethanol, isopropanol, ethylene glycol, propylene glycol, or a combination thereof.

The pharmaceutical compositions can be prepared, packaged, and/or sold in bulk, as a single unit dose, and/or as a plurality of single unit doses. A “unit dose” is a discrete amount of the composition comprising a predetermined amount of the therapeutic agents. The amount of the therapeutic agents is generally equal to the dosage of the therapeutic agents which would be administered to a subject and/or a convenient fraction of such a dosage, such as, for example, one-half, one-third, or one-quarter of such a dosage.

Relative amounts of the therapeutic agents, the excipient, and/or any additional ingredients in a composition of the disclosure will vary, depending upon the identity, size, and/or condition of the subject treated. By way of example, the composition may comprise between 0.1% and 99% (w/w), between 0.1% and 90% (w/w), between 0.1% and 80% (w/w), between 0.1% and 70% (w/w), between 1% and 50% (w/w), between 10% and 80% (w/w), between 10% and 90% (w/w), between 10% and 80% (w/w), between 20% and 80% (w/w), between 30% and 80% (w/w), between 30% and 70% (w/w), or between 40% and 60% (w/w), of the therapeutic agents.

Additional pharmaceutically acceptable excipients may be used in the manufacture of the provided pharmaceutical compositions. These include inert diluents, dispersing and/or granulating agents, surface-active agents and/or emulsifiers, disintegrating agents, binding agents, preservatives, buffering agents, lubricating agents, and/or oils. Excipients such as cocoa butter and suppository waxes, coloring agents, and coating agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80)), polyoxyethylene esters (e.g., polyoxyethylene monostearate (MYRJ 45), polyoxyethylene hydrogenated castor oil, polyethoxylated castor oil, polyoxymethylene stearate, and Solutol), sucrose fatty acid esters, polyethylene glycol fatty acid esters (e.g., Cremophor™), polyoxyethylene ethers, (e.g., polyoxyethylene lauryl ether (BRIJ 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC F-68, Poloxamer-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS, PHENONIP, methylparaben, GERMALL 115, GERMABEN II, NEOLONE, KATHON, and EUXYL.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

In some embodiments, the pharmaceutical compositions of the present disclosure comprise a pharmaceutically acceptable salt. The term “pharmaceutically acceptable salt” refers to those salts which are, within the scope of sound medical judgment, suitable for use in contact with the tissues of humans and lower animals without undue toxicity, irritation, allergic response, and the like and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, non-toxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁-C4 alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

Liquid dosage forms (e.g., for parenteral administration) include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Dosage forms for topical and/or transdermal administration of a compound described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

Although the descriptions of pharmaceutical compositions provided herein are principally directed to compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

The pharmaceutical compositions provided herein are typically formulated in a size (e.g., volume) and weight appropriate for the intended use (e.g., surgical implantation) for ease of administration. It will be understood, however, that the total amount of the composition of the present disclosure will be decided by the attending clinician or physician within the scope of sound medical judgment. The specific therapeutically effective dose level for any particular subject will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; the drugs used in combination or coincidental with the specific active ingredient employed; and like factors well known in the medical arts.

As described herein, the compositions of the present disclosure can also be administered in combination with one or more additional pharmaceutical agents. For example, the compositions can be administered in combination with additional pharmaceutical agents that reduce and/or modify their metabolism, inhibit their excretion, and/or modify their distribution within the body. It will also be appreciated that the additional therapy employed may achieve a desired effect for the same disorder, and/or it may achieve different effects.

The compositions can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Each additional pharmaceutical agent may be administered at a dose and/or on a time schedule determined for that pharmaceutical agent. The additional pharmaceutical agents will be administered separately in different doses and/or different routes of administration. The particular combination to employ in a regimen will take into account compatibility of the pharmaceutical composition with the additional pharmaceutical agents and/or the desired therapeutic and/or prophylactic effect to be achieved. In general, it is expected that the additional pharmaceutical agents utilized in combination be utilized at levels that do not exceed the levels at which they are utilized individually. In some embodiments, the levels utilized in combination will be lower than those utilized individually.

Exemplary additional pharmaceutical agents include, but are not limited to, anti-proliferative agents, anti-cancer agents, anti-inflammatory agents, immunosuppressant agents, and pain-relieving agents. Pharmaceutical agents include small molecule therapeutics such as drug compounds (e.g., compounds approved by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells.

### Administering

As used herein, the terms “administer,” “administering,” or “administration” refer to implanting, absorbing, ingesting, injecting, inhaling, or otherwise introducing a composition as described herein to a subject. Administering can involve any one of the modes of administration disclosed herein or a combination thereof.

### Treating

As used herein, the terms “treatment,” “treat,” and “treating” refer to reversing, alleviating, delaying the onset of, or inhibiting the progress of a “pathological condition” (e.g., a disease, disorder, or condition, including one or more signs or symptoms thereof) described herein. In some embodiments, treatment may be administered after one or more signs or symptoms have developed or have been observed. Treatment may also be continued after symptoms have resolved, for example, to delay or prevent recurrence and/or spread.

### Therapeutically Effective Amount

The present disclosure provides methods for treating a subject having cancer comprising administering a therapeutically effective amount of a stimulator of interferon signaling and a therapeutically effective amount of a heparin polysaccharide.

A “therapeutically effective amount” (also referred to as an effective amount) is a dose sufficient to provide a medically desirable result and can be determined by one of skill in the art using routine methods. In some embodiments, an effective amount is an amount which results in any improvement in the condition being treated. In some embodiments, an effective amount may depend on the type and extent of the disease or condition being treated and/or use of one or more additional therapeutic agents. However, one of skill in the art can determine appropriate doses and ranges of therapeutic agents to use, for example based on in vitro and/or in vivo testing and/or other knowledge of compound dosages.

When administered to a subject, effective amounts of the therapeutic agent will depend, of course, on the particular disease being treated; the severity of the disease; individual patient parameters including age, physical condition, size and weight, concurrent treatment, frequency of treatment, and the mode of administration. These factors are well known to those of ordinary skill in the art and can be addressed with no more than routine experimentation. In some embodiments, a maximum dose is used, that is, the highest safe dose according to sound medical judgment.

In the treatment of a subject having cancer, an effective amount is that amount which slows the progression of the cancer (e.g., the growth of the tumor—as determined by size, metastasis), halts the progression of the disease, or reverses the progression of the disease. An effective amount includes that amount necessary to slow, reduce, inhibit, ameliorate or reverse one or more symptoms associated with the cancer. Disease progression can be monitored by clinical observations, laboratory and imaging investigations apparent to a person skilled in the art. A therapeutically effective amount can be an amount that is effective in a single dose or in a multi-dose therapy (e.g., an amount that is administered in two or more doses or administered chronically).

Chronic treatments include forms of repeated administration for an extended period of time (e.g., for one or more months, between a month and a year, one or more years, or longer). In many embodiments, a chronic treatment involves administering the compositions of the present disclosure repeatedly over the duration of illness of the patient. In general, a suitable dose such as a daily dose of a structure described herein will be that amount of the structure that is the lowest dose effective to produce a therapeutic effect. Such an effective amount will generally depend upon the factors described above.

### Local or Intratumoral Administration

In some embodiments of the present disclosure, the therapeutically effective amount of a stimulator of interferon signaling and therapeutically effective amount of a heparin polysaccharide are administered locally. Local administration targets a specific tissue, organ, or body part would be at the site of the tumor. The term “local” refers to administration of the agent(s) either within or in close proximity to the site of cancer or tumor such that, when administered, the agent(s) selectively affects the targeted cancer or tumor. This is in contrast with systemic administration, which involves dissemination of the agent(s) throughout the body.

As used herein, the term “close proximity” refers to a distance of no more than 2 cm and more preferably no more than 1 cm away from the tumor (e.g., outermost cells of the tumor). In some embodiments, close proximity refers to a distance of 0.1, 0.2, 0.3, 0.4, 0.5, 0.6, 0.7, 0.8, 0.9 or 1 cm away from the tumor. In some embodiments, close proximity refers to a distance of 0.0-0.2, 0.0-0.4, 0.0-0.6, 0.0-0.8, 0.0-0.9, 0.2-0.4, 0.2-0.6, 0.2-0.8, 0.2-0.9, 0.4-0.6, 0.4-0.8, 0.4-0.9, 0.5-0.8, 0.5-0.9, 0.6-0.8, 0.6-0.9, 0.7-0.8, 0.7-0.9, 0.8-0.9, 0.8-0.95, 0.9-0.95, or 0.9-1.0 cm from the tumor.

In some embodiments of the present disclosure, the therapeutically effective amount of a stimulator of interferon signaling and therapeutically effective amount of a heparin polysaccharide are administered locally. In some embodiments, local administration refers to “intratumoral administration,” which refers to the administration of the agent(s) inside of the tumor (see, for example, Marabelle, Aurdlien, et al. (Annals of Oncology 29.11 (2018): 2163-2174), the relevant disclosures of which are herein incorporated by reference. This can be done in an effective amount to treat the tumor and not the surrounding areas.

The compositions of the present invention can be administered by any available or effective delivery method. Delivery methods include, but are not limited to, intravenously, intradermally, intraarterially, intralesionally, intratumorally, intracranially, intraarticularly, intraprostaticaly, intrapleurally, intratracheally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, transdermal drug delivery, injection, infusion, continuous infusion, localized perfusion bathing target cells directly, via a catheter, via a lavage, in creams, in lipid compositions (e.g., liposomes), or by other method or any combination of the forgoing as would be known to one of ordinary skill in the art (see, for example, Remington's Pharmaceutical Sciences (1990), incorporated herein by reference). The mode of administration used may depend on the type of cancer. A person of ordinary skill would be able to determine the appropriate mode of administration for a subject.

In some cases, the delivery of the present invention can utilize polymers that can either alter, slow, or pulsate the release of the composition, including but not limited to microparticles, including engineered polyactic-co-glycolic acid (PLGA) microparticles (see, e.g., Lu et. al, Engineered PLGA microparticles for long-term, pulsatile release of STING agonists for cancer immunotherapy, Sci. Transl. Med., 12: eaaz6606 (Aug. 12, 2020); and self-assembled hydrogels (Wang et al., Tumour sensitization via the extended intratumoural release of a STING agonist and camptothecin from a self-assembled hydrogel, Nature Biomedical Engineering, https://doi.org/10.1038/s41551-020-0597-7 (August 2020). In some embodiments, the polymer can be used to deliver either the heparin polysaccharide, the stimulator of interferon signaling, or both.

Intratumoral administration in some cases leads to rapid diffusion of the drug from the site of the tumor and reduced effectiveness of the drug at the site of administration. In certain embodiments, the drug with the longer half-life is administered intratumorally. In some embodiments, the heparin polysaccharide and/or the stimulator of interferon signaling is formulated for prolonged efficacy.

It should be understood that the mode of administration for the heparin polysaccharide need not be the same mode of administration for the stimulator of interferon signaling. For example, in some embodiments, the stimulator of interferon signaling may be administered intratumorally while the heparin polysaccharide is administered by IV infusion.

### Times of Administration

As disclosed herein, the heparin polysaccharide and the stimulator of interferon signaling can be administered at the same time. If administered separately, the term “at the same time” may encompass administration of the heparin polysaccharide and the stimulator of interferon signaling within about 1, 2, 3, 4, 5, 6, 7, 8, 9, or 10 minutes or less of each other. Alternatively, the heparin polysaccharide may be administered before the stimulator of interferon signaling. Alternatively, the heparin polysaccharide may be administered after the stimulator of interferon signaling. If not administered at the same time, administration can be within 1 day of each other. In some embodiments, the administration can be within about 0.5, 1, 1.5, 2, 2.5, 3, 3.5, 4, 4.5, 5, 6, 7, 8, 9, 10, 11, 12, 13, 14, 15, 16, 17, 18, 19, 20, 21, 22, 23, or 24 hours of each other.

In certain embodiments, for a single dosing, the heparin polysaccharide and the stimulator of interferon signaling, are administered within the half-life of either drug in the tumor. The half-life of heparin polysaccharide is dependent on the type of heparin molecule. For example, some unfractionated heparin molecules are known to have a half-life of 1-2 hours. In contrast, some low molecular weight heparin molecules are known to have a half-life of 4-5 hours. The administration should be close enough in time (whether by the same or different routes) such that the beneficial and synergistic effects of the heparin on the STING agonist may be realized.

### Subject

As used herein, a “subject” or a “patient” refers to any mammal (e.g., a human), for example, a mammal that may be susceptible to a disease or bodily condition such as a disease or bodily condition that is, for instance, a vascular condition, disease or disorder (e.g., ischemia reperfusion injury after organ transplant). Examples of subjects or patients include a human, a non-human primate, a cow, a horse, a pig, a sheep, a goat, a dog, a cat or a rodent such as a mouse, a rat, a hamster, or a guinea pig. In certain embodiments, a subject may be selected for treatment on the basis of a known disease or bodily condition in the subject. A subject may be a subject diagnosed with a certain disease or bodily condition or otherwise known to have a disease or bodily condition. In some embodiments, a subject may be diagnosed as, or known to be, at risk of developing a disease or bodily condition. In certain embodiments, a subject may be diagnosed with a tumor (malignant or benign).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1

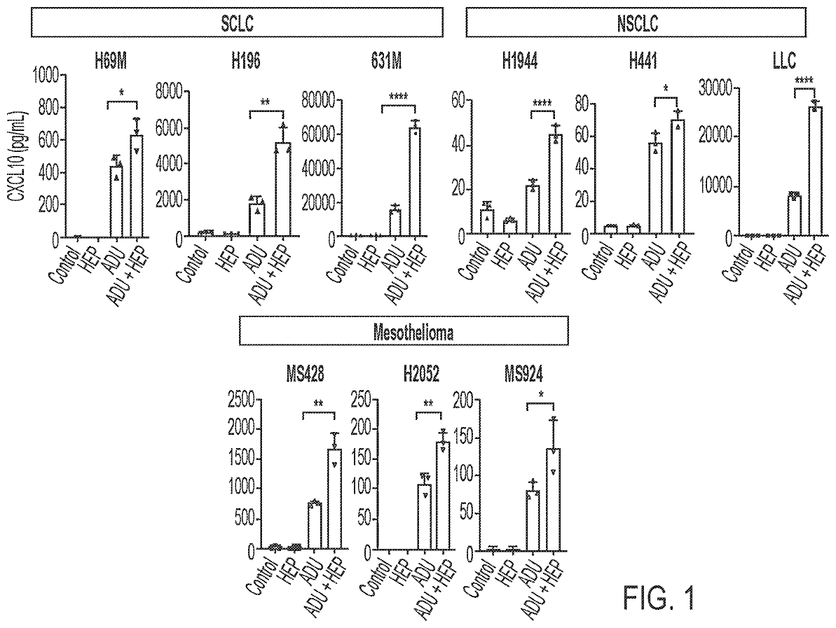

Heparin was found to enhance STING agonist activity in cancer cells. Human and mouse cancer cell lines were treated with 50 μM ADU S-100+/−heparin at a concentration of 10 μg/mL (human cells) or 1 μg/mL (mouse cells) for 24 hours prior to conditioned media collection for CXCL10 ELISA (FIG. 1). The ELISA results for all cell lines showed that the coadministration of heparin and ADU S-100 yielded significantly higher STING activity (as indicated by the amount of CXCL10 in the media) than the administration of ADU 5-100 alone.

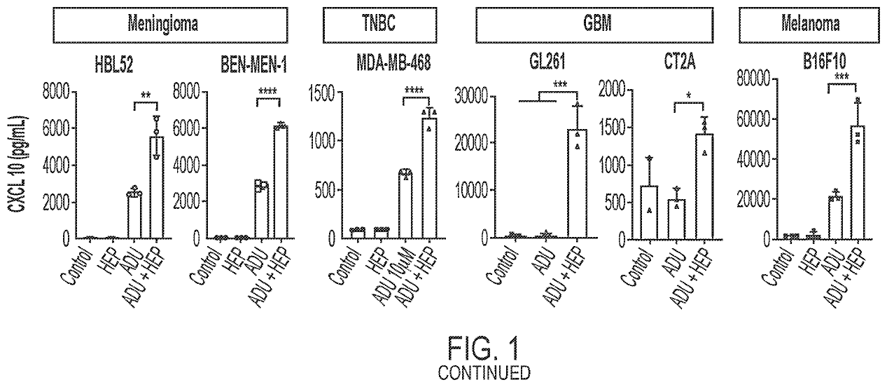

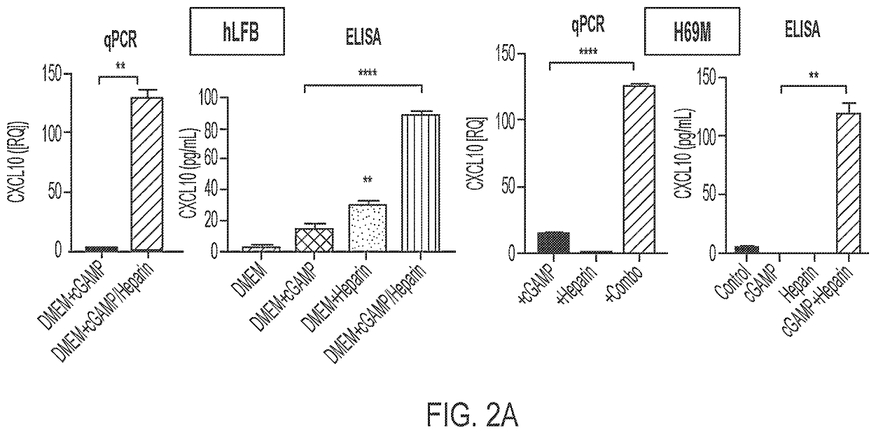

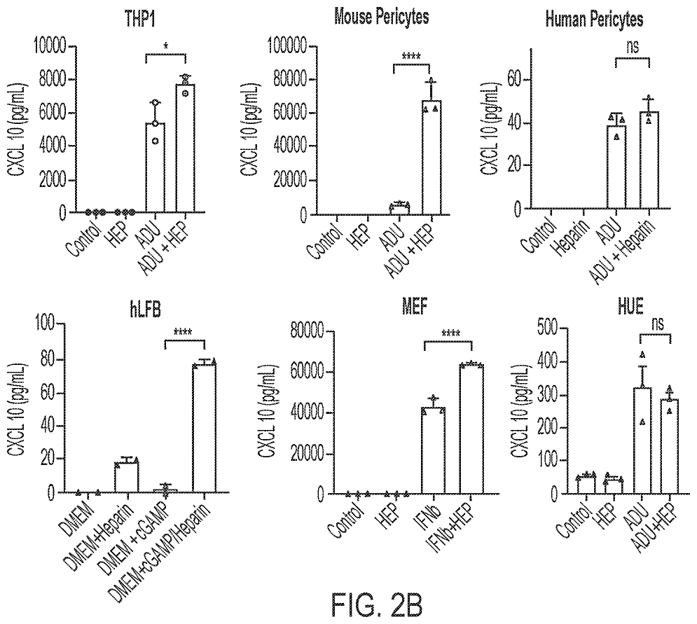

Also, Human lung fibroblasts (hLFB) were treated with 2,3-cGAMP 1 μg/mL (hereafter referred to as “cGAMP)+/−heparin 1 μg/mL for 24 hours prior to CXCL10 qPCR and collection of conditioned media for CXCL10 ELISA (FIG. 2A). The qPCR results showed that the treatment of hLFB cells with cGAMP in the absence of heparin yielded negligible STING activity, as indicated by CXCL10 expression. However, the addition of heparin resulted in significantly enhanced STING activity, as indicated by CXCL10 expression (p<0.01). The ELISA results for hLFB cells showed that the coadministration of heparin and cGAMP yielded significantly higher STING activity (as indicated by CXCL10 concentration) than the administration of cGAMP alone (DMEM+cGAMP) (p<0.0001). Additionally, the administration of heparin with DMEM resulted in significantly enhanced STING activity compared with the negative control (DMEM alone)(p<0.01), as indicated by CXCL10 expression (FIG. 2A).

In addition, small cell lung cancer H69M cells were treated with 2,3-cGAMP 1 μg/mL (hereafter referred to as “cGAMP)+/−heparin 1 μg/mL for 24 hours prior to CXCL10 qPCR and collection of conditioned media for CXCL10 ELISA. The qPCR and ELISA results showed that the treatment of H69M cells with cGAMP and heparin (FIG. 2A, “combo”) yielded significantly enhanced STING activity, as indicated by CXCL10 expression (p<0.0001 for qPCR; p<0.01 for ELISA). There was no significant difference in CXCL10 expression between treatment with cGAMP alone and treatment with heparin alone (FIG. 2A).

Heparin was also found to enhance STING agonist activity in benign immortalized cell lines. Human and mouse immortalized cell lines were also treated with 50 μM ADU S-100, 10 μg/mL 2′3′-cGAMP (hereinafter cGAMP), or 1 ng/mL IFN-beta (IFNb)+/−heparin at a concentration of 10 μg/mL (human cells) or 1 μg/mL (mouse cells) for 24 hours prior to conditioned media collection for CXCL10 ELISA (FIG. 2B). The ELISA results for most cell lines showed that the coadministration of heparin and ADU S-100 yielded significantly higher STING activity (as indicated by amount of CXCL10) than the administration of ADU S-100 alone. The only exceptions were for Human Pericytes and Human Umbilical Endothelial Cells, where no significant difference was observed.

### Example 2

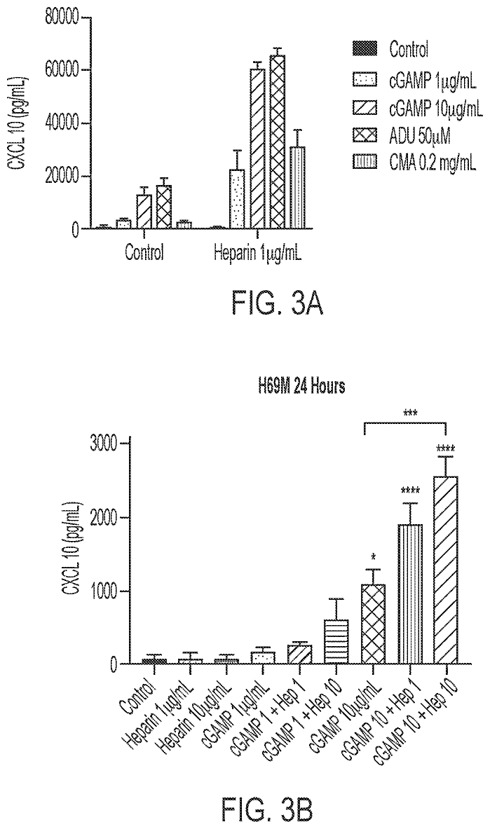

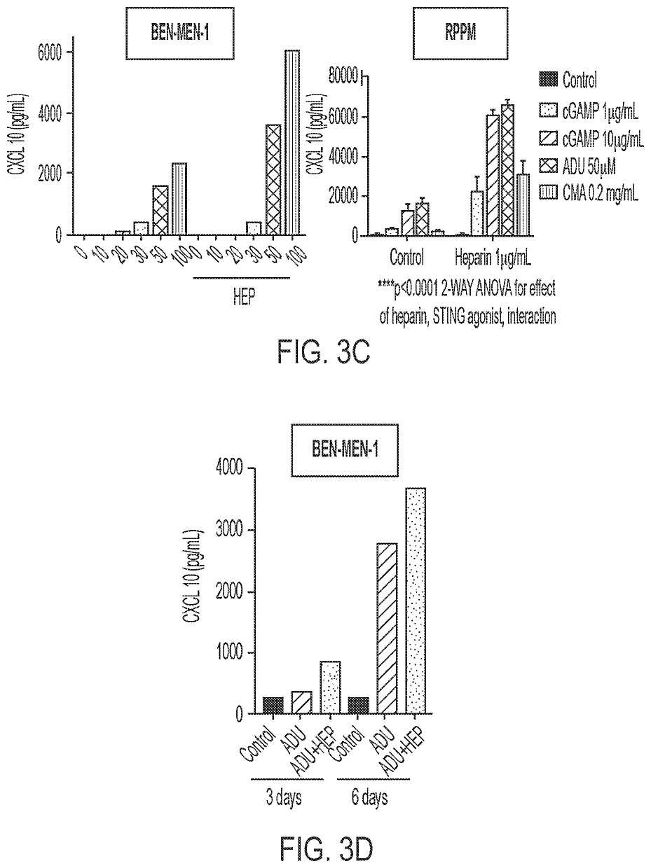

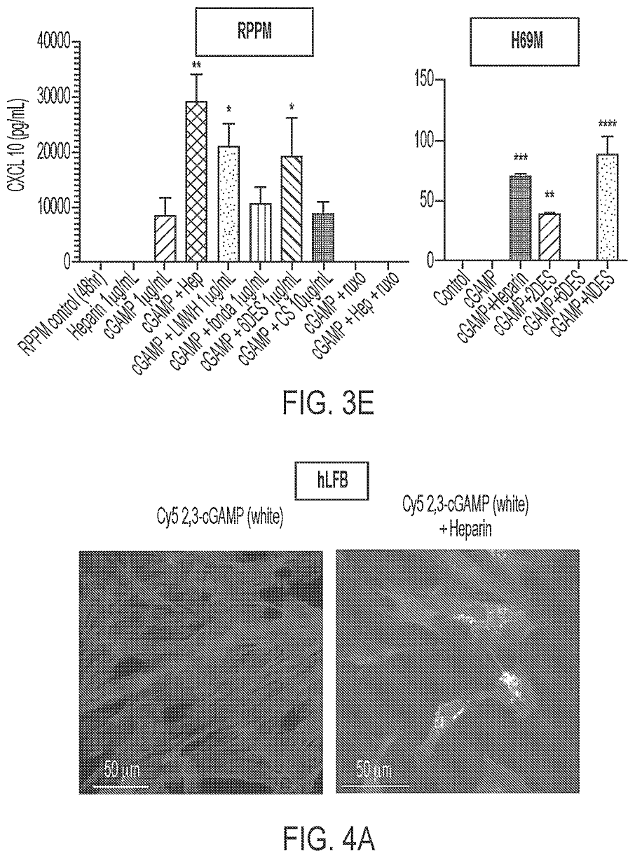

Heparin was found to dose-dependently enhance STING agonists effects across various STING agonists. 631M/RPPM mouse SCLC cells were treated for 24 hours either with or without 1 μg/mL heparin and the following STING agonists: 1 μg/mL cGAMP, 10 μg/mL cGAMP, 50 μM ADU, or 0.2 mg/ml CMA. The CXCL10 ELISA results showed a significant interaction between heparin and all of the STING agonists (cGAMP, ADU, CMA) on the amount of CXCL10 in the media, which was not observed with the control. (FIG. 3A).

An additional 24-hour dose response study was conducted on H69M cells. The addition of 1 μg/mL of heparin to either 1 μg/mL cGAMP or 10 μg/mL cGAMP significantly increased STING activity, as indicated by amount of CXCL10 in the media. Compared to administering cGAMP alone, STING activation (as indicated by amount of CXCL10 in the media) was shown to increase significantly with heparin (FIG. 3B).

A 24-hour dose response study was also conducted on BEN-MEN-1 meningioma cells (hereafter “BenMen 1 cells”) and RPPM mouse SCLC cells. BenMen 1 cells were treated with various doses of ADU S-100 (0, 10, 20, 30, 50, and 100 μg/mL) either in the presence or absence of heparin (10 μg/mL). The results showed that 50 μg/mL ADU-S100 and 100 μg/mL ADU-S100 yielded significantly higher STING activity, as indicated by CXCL10 concentration, in the presence of heparin (FIG. 3C). A time course with treatment of BenMen 1 cells for 3 and 6 days showed that with time, the effect of coadministering the STING is more pronounced (there is significantly greater STING activity, as indicated by CXCL10 concentration) (FIG. 3D). RPPM primary mouse SCLC cells were treated for 24 hours with STING agonists 2,3-cGAMP, ADU-S100, and CMA. The CXCL10 ELISA results showed a significant interaction between heparin and STING agonists (cGAMP, ADU, CMA) (p<0.0001 by 2-WAY ANOVA) (FIG. 3C). Time course data (24 h, 48 h, and 72 hour treatment) revealed similar patterns across the cell lines (DATA NOT SHOWN).

### Example 3

RPPM mouse SCLC cells were treated with 1 μg/mL 2,3-cGAMP and 1 μg/mL unfractionated heparin, low-molecular weight heparin (LMWH), heparin pentasaccharide fondaparinux, 6-desulfated heparin, chondroitin sulfate+/−the JAK/STAT inhibitor ruxolitinib (ruxo 1 μg/mL) for 24 hours prior to CXCL10 ELISA. H69M human SCLC cells were treated with 10 μg/mL 2,3-cGAMP or 50 μM ADU+/−heparin 10 μg/mL or desulfated heparins heparins 2-O desulfated (2DES), N-desulfated (NDES), and 6-O desulfated (6DES) 24 hours prior to CXCL10 ELISA. Low molecular weight heparin and some desulfated heparins were shown to significantly enhance STING activity, as indicated by CXCL10 release, in a similar fashion to unfractionated heparin, but fondaparinux did not. Chondroitin sulfate was also added as a negative control, confirming that the heparan sulfate is unique among glycosaminoglycans (FIG. 3E).

### Example 4

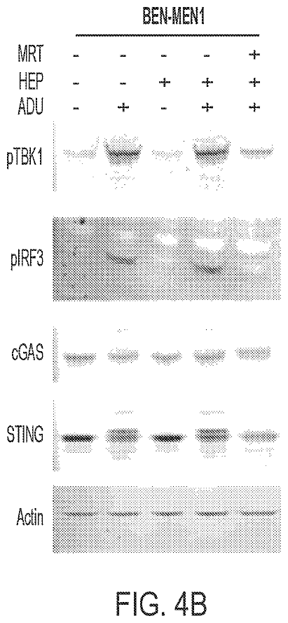

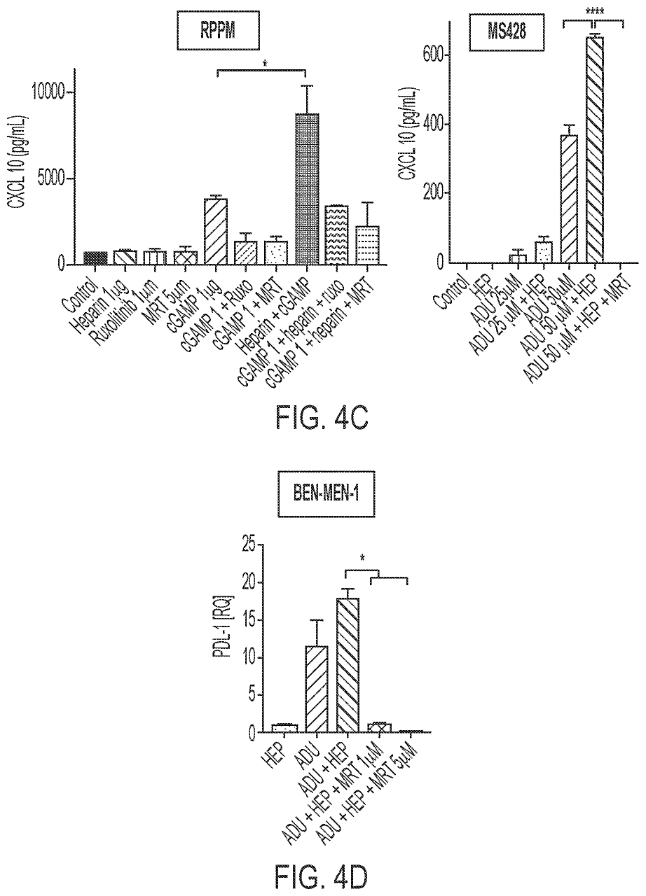

Immunofluorescent staining was used to visualize the localization of STING agonists and heparin in the cells. Briefly, hLFB cells were treated with Cy5-labeled 2,3-cGAMP 1 μg/mL in the presence or absence of heparin (1 μg/mL). The fluorescent images of cells treated with both heparin and Cy5-tagged STING agonist (e.g., cGAMP, stained white) showed dense white punctae within the cell, localizing proximal to the nuclei. These white punctae were not visible when the cells were treated with STING agonist in the absence of heparin. This indicated that the treatment of cells with both heparin and the STING agonist results in localization of the STING agonist within the cells, contributing to the enhanced STING activation (FIG. 4A).

These results indicate that heparin increases STING agonist uptake in cancer cells.

### Example 5

The serine/threonine protein kinase, TBK1, is a critical regulator in innate immune signaling pathways that lead to the induction of type I interferon (IFN) and interferon-stimulated genes (ISGs). Dysregulation of TBK1 activity is often associated with autoimmune diseases and cancer. Herein, BenMen 1 cells were treated for 72 hours with 50 μM ADU S100 (herein referred to as “ADU”) in the presence or absence of heparin 10 μg/mL and 5 μM MRT TBK1 inhibitor was administered to cells receiving both ADU and heparin. Then CXCL10 ELISA was conducted after 24 hours treatment with 50 μM ADU+/−heparin 10 μg/mL and MRT TBK1 inhibitor 1 μM or 5 μM or JAK/STAT inhibitor ruxolitinib 1 μM in the indicated cell lines. As shown in FIGS. 4B-4C, the combination of heparin and STING agonist (e.g., ADU) results in significantly enhanced STING activity (as indicated by CXCL10 intensity or concentration), and the addition of MRT TBK1 inhibitor or ruxolitinib STAT inhibitor reduced the STING activity. Similar results were also shown for mesothelioma cell line MS428 (FIGS. 4B-C).

PDL-1 expression in BenMen 1 cells was examined using qPCR after 24 hours treatment with 50 μM ADU+/−heparin 10 μg/mL and MRT TBK1 inhibitor. The results showed increased PDL1 expression when ADU and heparin were coadministered (relative to heparin alone or STING agonist alone). Additionally, PDL-1 expression was significantly reduced when MRT TBK1 was added to the combination of STING agonist and heparin (p<0.05) (FIG. 4D).

These results indicated that heparin increases STING agonist activation of downstream signaling.

### Example 6

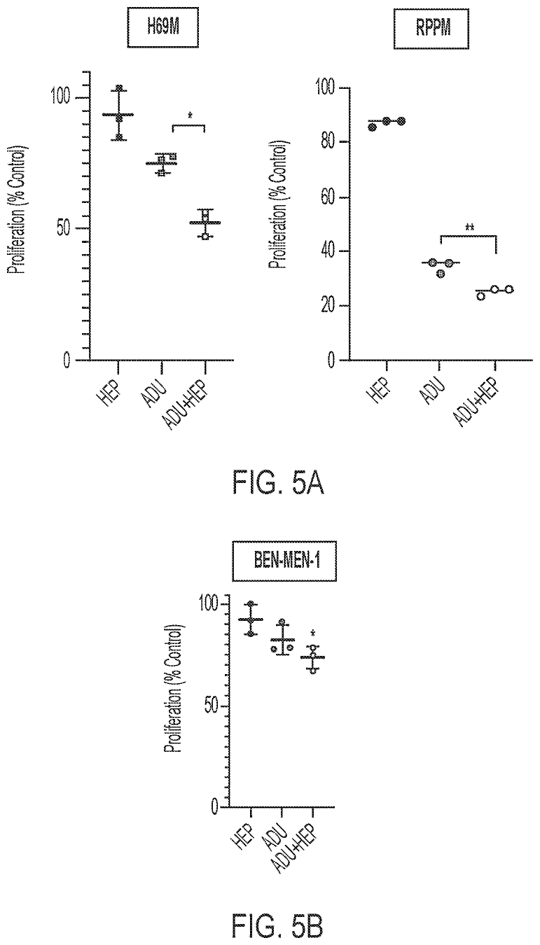

Heparin was found to increase STING agonist suppression of cancer cell growth in vitro. A cell-titer glow proliferation assay (CellTiter-Glo® Luminescent Cell Viability Assay) was used to assess the influence of heparin on cancer cell growth as determined by the amount of proliferation (either with or without a STING agonist). H69M Human SCLC cells, Benmen 1 meningioma cells, and 631M/RPPM mouse SCLC cells were evaluated following 24 hours of treatment with 50 μM ADU+/−heparin (1 μg/mL or 10 μg/mL). As shown in FIGS. 5A-5B, the proliferation percentage (relative to the negative control (no STING agonist or heparin)) of cells treated with ADU and heparin was significantly lower than that of cells treated with ADU alone (p<0.05 for H69M cells; p<0.05 for Benmen 1 cells; p<0.01 for RPPM cells). These results indicated that heparin increases STING agonist suppression of cancer cell growth.

### Example 7

NF-κB is a protein complex that is widely used by eukaryotic cells and controls transcription of DNA, cytokine production and cell survival. Many different types of human tumors have misregulated NF-κB (i.e. NF-κB is constitutively active). IL-6 and IL-8 are examples of NF-κB-associated cytokines. IL-8 is an example of a growth-promoting cytokine.

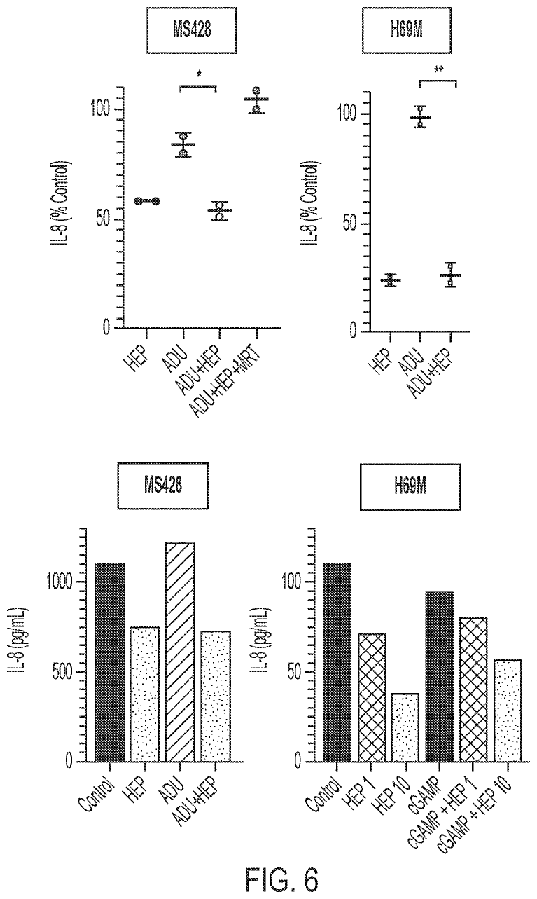

Herein, the influence of heparin (with and without STING agonist) on IL-8 levels was examined. In both MS428 meningioma cells and H69M SCLC cells, the IL-8 levels (% relative to negative control (no STING agonist and no heparin)) after 24 hours of treatment with 50 μM ADU and heparin 10 μg/mL was significantly reduced compared to IL-8 levels after 24 hours of treatment with ADU alone (p<0.05 for MS428 cells; p<0.01 for H69M cells). In MS428 cells, the addition of MRT TBK1 inhibitors was shown to increase IL-8 levels relative to heparin combined with STING agonist (e.g., ADU) and relative to STING agonist alone (e.g., ADU alone). In H69M cells, as the concentration of heparin increased, the reduction of IL-8 levels was more pronounced (FIG. 6). Overall, the results indicated that heparin inhibits NF-κB-associated cytokine release after STING agonist treatment.

### Example 8

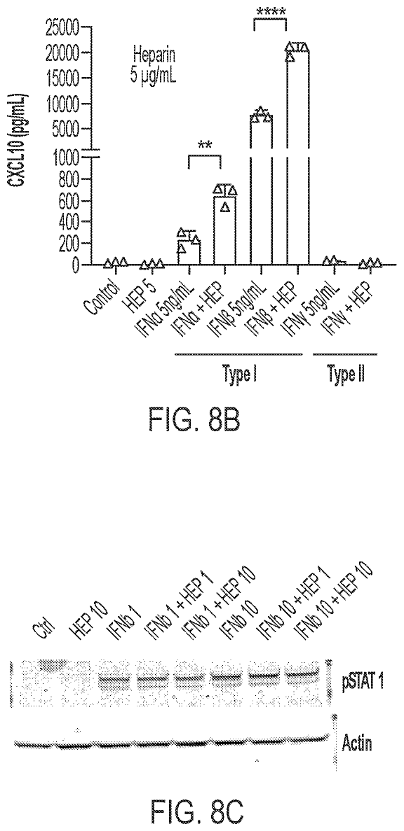

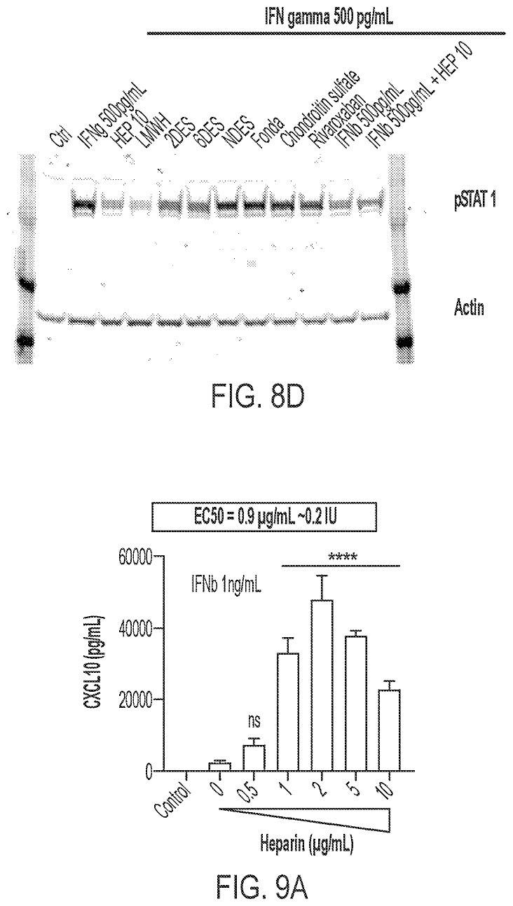

Heparin was found to enhance type 1 interferon effects, but did not similarly enhance the effects of interferon gamma. B16F10 mouse melanoma cells lines were treated with interferon alpha (IFNa), interferon beta (IFNb), or interferon gamma (IFNg) (5 ng/mL)+/−heparin (1 μg/mL) for 24 hours prior to conditioned media collection for CXCL10 ELISA (FIG. 8A). Lewis Lung Carcinoma (LLC) mouse non-small-cell lung cancer cells were treated with interferon alpha (IFNa), interferon beta (IFNb), or interferon gamma (IFNg) (5 ng·mL)+/−heparin (1 μg/mL) for 24 hours prior to conditioned media collection for CXCL10 ELISA (FIG. 8B). Compared to administering IFNa or IFNb alone, the amount of CXCL10 in the media was shown to increase significantly with heparin. This effect was not seen with IFNg.

B16F10 cells were treated for six hours with either 1 ng/mL or 10 ng/mL IFNb+/−heparin (at a concentration or either 1 μg/mL or 10 μg/mL). Western blot assay for pSTAT1 and beta-actin load (control) indicated that heparin had no effect on the amount of pSTAT1 protein, indicating that heparin does not act to enhance the canonical JAK/STAT signaling pathway. (FIG. 8C).

In contrast, heparin and some modified forms of heparin suppressed the activity of IFNg activity in cancer cells. H69M Human SCLC cells were treated for 30 minutes with 500 pg/ml IFNg and various forms of heparin (1 μg/mL). Western blot assay for pSTAT1 and beta-actin load (control) indicated that heparin and some modified forms decreased the amount of pSTAT1 when coadministered with IFNg. (FIG. 8D)

### Example 9

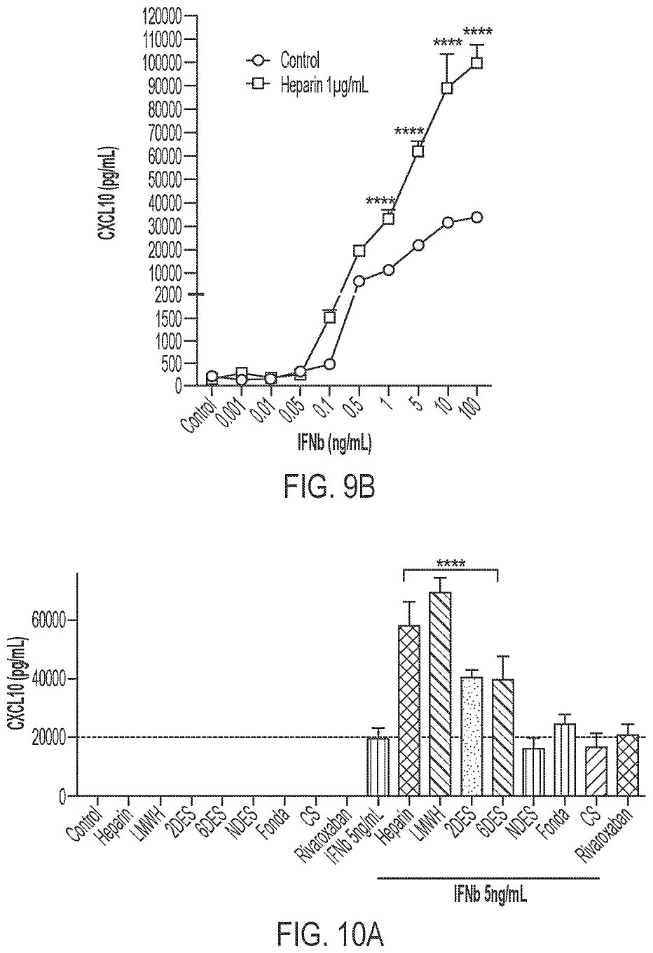

Heparin's effect on IFNb was found to be dose dependent. A 24-hour dose response study were conducted on B16F10 mouse melanoma cells. Cells were treated with various doses of heparin (0, 0.5, 1, 2, 5, and 10 μg/mL) either in the presence or absence of IFNb (1 ng/mL). The results showed that 1 μg/mL, 2 μg/mL 5 μg/mL, and 10 μg/mL of heparin significantly increased the effect of IFNb on the amount of CXCL10 in the media, (FIG. 9A) Cells were also treated with various doses of IFNb either in the presence or absence of heparin (1 μg/mL) (FIG. 9B). The results showed that 1 ng/mL, 5 ng/mL, 10 ng/mL, and 100 ng/mL of IFNb in the presence of heparin yielded significantly higher amounts of CXCL10 in the culture media.

### Example 10

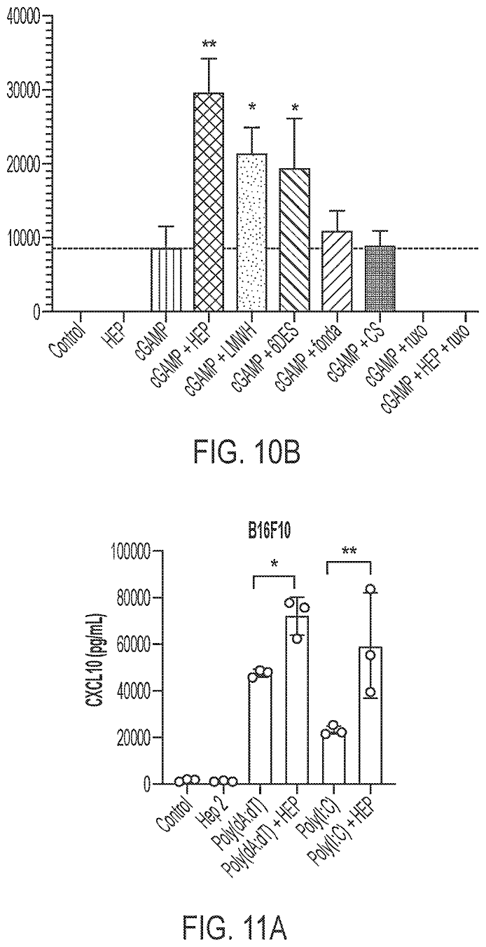

Modified heparins were also found to enhance the effects of IFNb and STING agonists. B16F10 mouse melanoma cells were treated with 5 ng/mL IFNb+/−1 μg/mL of various forms of heparin, including unfractionated heparin, low-molecular weight heparin (LMWH), 2- and 6-, and Ndesulfated heparin (2DES, 6DES, NDES), and the heparin pentasaccharide fondaparinux, as well as controls including chondroitin sulfate (CS) and rivaroxaban. A CXCL10 ELISA from conditioned media was run after 24 hours of treatment (FIG. 10A). Low molecular weight heparin and some desulfated heparins were shown to significantly enhance IFNb activity, as indicated by CXCL10 release, in a similar fashion to unfractionated heparin, but fondaparinux did not. The use of Chondroitin sulfate as a control showed that heparan sulfate is unique among glycosaminoglycans.

RPPM mouse SCLC cells were treated with 1 μg/mL of the STING agonist cGAMP and 1 μg/mL various forms of heparin, including unfractionated heparin, low-molecular weight heparin (LMWH), heparin pentasaccharide fondaparinux, 6-desulfated heparin, chondroitin sulfate. In addition, cells were treated with cGAMP and +/−1 μg/mL of the JAK/STAT inhibitor ruxolitinib (ruxo). A CXCL10 ELISA from conditioned media was run after 24 hours of treatment (FIG. 10B). Low molecular weight heparin and 6DES heparins were shown to significantly enhance STING activity, as indicated by CXCL10 release, in a similar fashion to unfractionated heparin, but fondaparinux did not. Chondroitin sulfate as a control showed that heparan sulfate is unique among glycosaminoglycans.

### Example 11

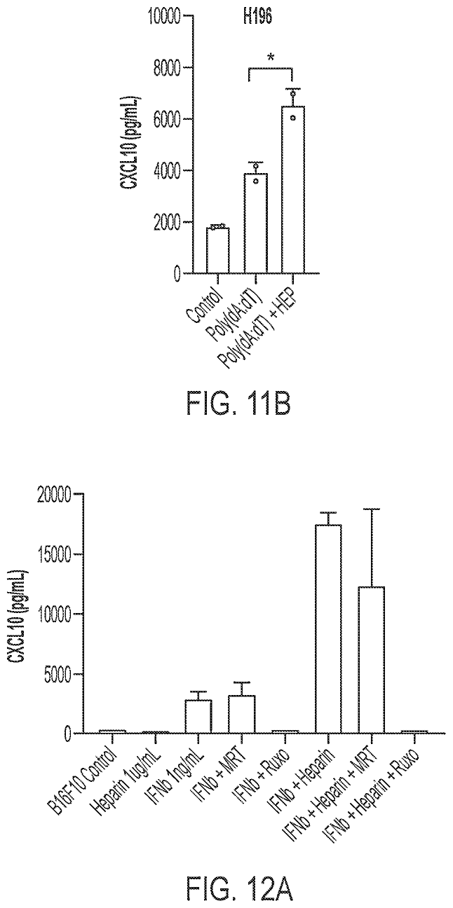

Heparin was found to enhance CXCL10 downstream of multiple inflammatory stimuli. B16F10 mouse melanoma cell lines were transfected with 1 g Poly(dA:dT) or Poly(I:C) for 4 hours followed by treatment with either 1 μg/mL heparin or control for 24 hours. A CXCL10 ELISA from conditioned media was run (FIG. 11A). Heparin was shown to increase significantly the amount of CXCL10, as measured in the media, when compared to transfecting with Poly(dA:dT) or Poly(I:C) alone.

H196 human SCLC cell lines were also transfected with 1 g Poly(dA:dT) for 4 hours followed by treatment with either 10 μg/mL heparin or control for 24 hours. A CXCL10 ELISA from conditioned media was run (FIG. 11B). Heparin was also shown to significantly increase the amount of CXCL10, as measured in the media, when compared to transfecting with Poly(dA:dT) alone.

### Example 12

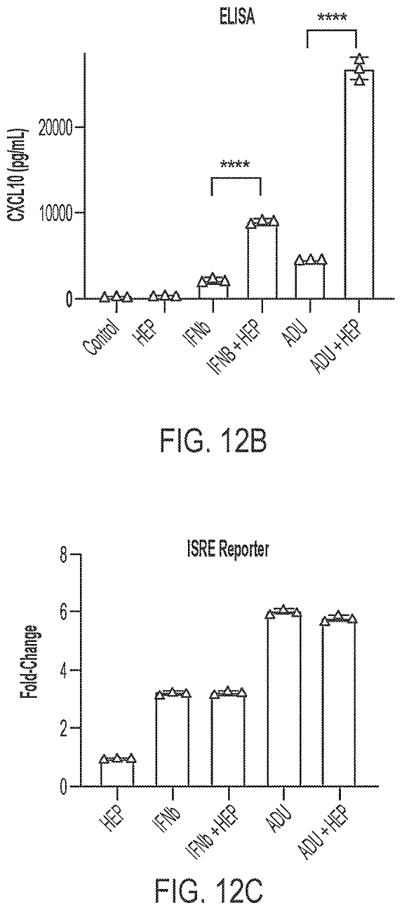

Heparin was found to not enhance canonical JAK/STAT signaling. B16F10 mouse melanoma cell lines were treated with 1 ng/mL IFNb+/−1 μg/mL heparin and either MRT67307 (MRT) or Ruxolitinib (ruxo) for 24 hours. A CXCL10 ELISA from conditioned media was run (FIG. 12A). As with before, Heparin was shown to significantly increase the amount of CXCL10, as measured in the media, when compared to use of IFNb alone. A similar result was also seen with the cells treated with MRT, a TBK1 inhibitor. However, CXCL10 release was suppressed by Ruxo, a JAK1 inhibitor, and this suppression was not affected by Heparin.

Heparin was also found to not enhance ISRE binding. B16 Blue cell lines were treated with either 500 μg/mL IFNb or 50 μM ADU-S100+/−5 μg/mL heparin for 24 hours. A CXCL10 ELISA from conditioned media was run (FIG. 12B). Heparin was shown to significantly increase the amount of CXCL10, as measured in the media, when compared to use of IFNb alone or ADU-S100 alone. However, heparin had no effect on gene expression, either with IFNb or ADU-S100, based on an ISRE chromogenic reporter assay (used according to the manufacturer's instructions) (FIG. 12C).

### Example 13

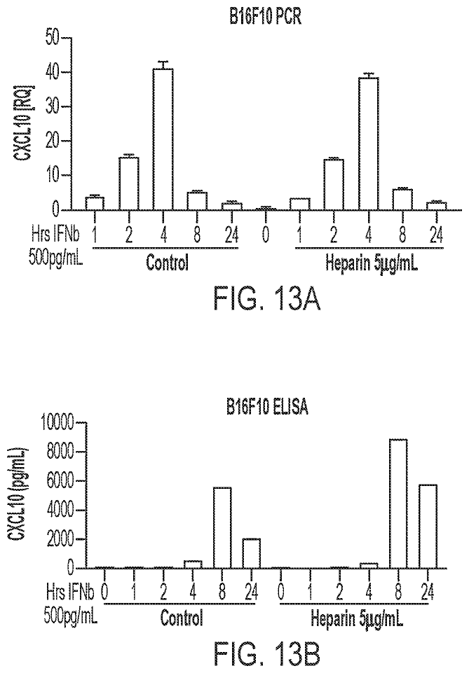

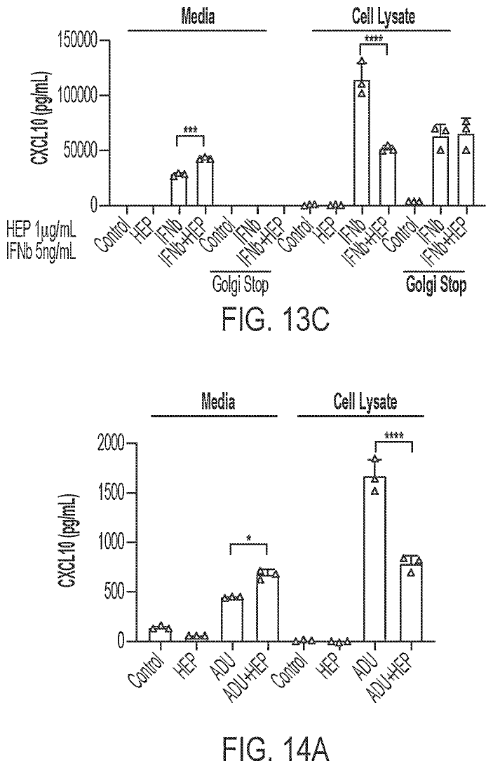

Heparin effect on IFNb were found to be time dependent. B16F10 mouse melanoma cell lines were treated with 500 μg/mL IFNb+/−5 μg/mL heparin for 24 hours. Both a quantitative RT-PCR reaction to measure CXCL10 mRNA levels (FIG. 13A) and a CXCL10 ELISA to measure the amount of CXCL10 released from the cell were run (FIG. 13B). The effect of heparin on IFNb signaling were found to be time dependent. Moreover, heparin did not have much influence on the mRNA levels of CXCL10.

Heparin was found to enhance CXCL10 release from cells treated with IFNb. B16F10 mouse melanoma cell lines were treated with 5 ng/mL IFNb+/−1 μg/mL heparin either with or without Golgi-Stop from BD biosciences for 6 hours. CXCL10 ELISAs from conditioned media and from cell lysate collections were run (FIG. 13C). As before, Heparin was shown to significantly increase the amount of CXCL10, as measured in the media, when compared to use of IFNb alone. However, the amount of CXCL10 in cell lysate collection was decrease when cells were treated with heparin and IFNb as compared to IFNb alone. No change was seen when Golgi-Stop was used.

### Example 14

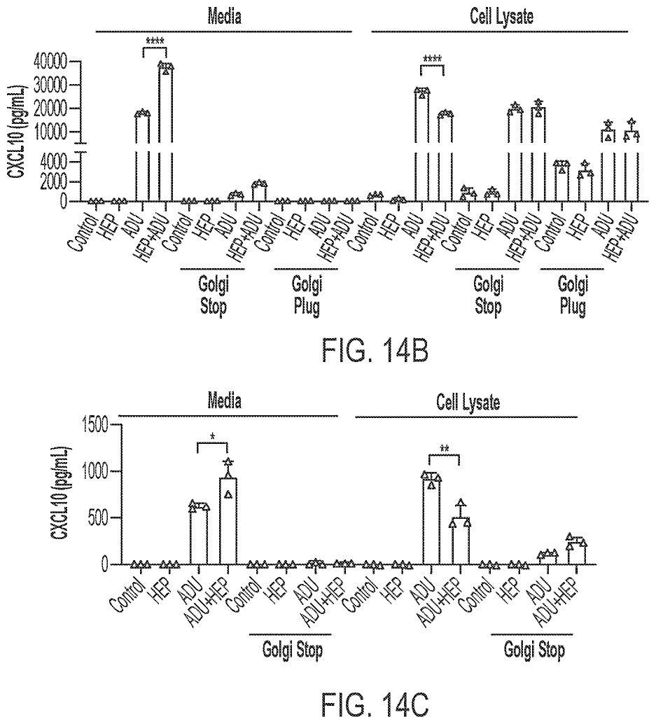

Heparin was found to enhance CXCL10 release from cells treated with STING agonists in both mouse and human cells. B16F10 mouse melanoma cell lines were treated with 50 μM ADU-S100+/−5 μg/mL heparin. A CXCL10 ELISA from conditioned media was run after 6 hours of treatment (FIG. 14A). Again, heparin was shown to significantly increase the amount of CXCL10, as measured in the media, when compared to use of ADU-S100 alone. However, the amount of CXCL10 in cell lysate collection was decreased when cells were treated with both heparin and ADU-S100 as compared to ADU-S100 alone.

A similar experiment was run on B16F10 mouse melanoma cell lines using 0.5 μL Golgi-Stop or Golgi-Plug from BD biosciences. A CXCL10 ELISA from conditioned media was run after 6 hours of treatment (FIG. 14B). Unlike the effect that heparin had on the administration of ADU-S100, there was little effect on the levels of CXCL10 in cell lysates when using Golgi-Stop and Golgi-Plug.

MS428 human mesothelioma cells were also treated with 50 μM ADU-S100+/−10 μg/mL heparin. 0.5 μL Golgi-Stop or Golgi-Plug from BD biosciences was also used. A CXCL10 ELISA from conditioned media was run after 12 hours of treatment (FIG. 14C). Similar to the mouse results, there was little effect on the levels of CXCL10 in cell lysates when using Golgi-Stop and Golgi-Plug unlike the effect that heparin had on the administration of ADU-S100.

### Example 15

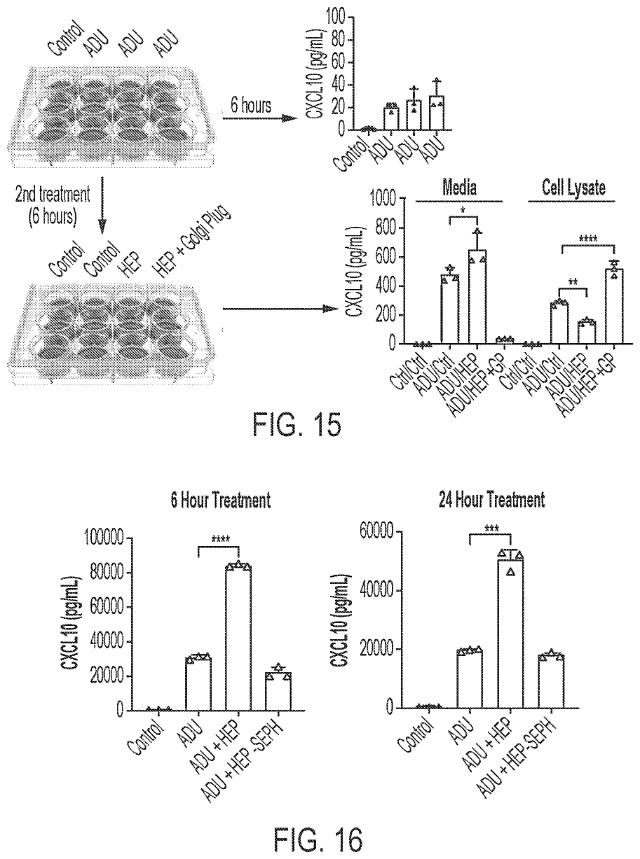

Heparin was found to enhance cytokine release in human mesothelioma cells over time. MS428 human mesothelioma cells were treated with 50 μM ADU-S100. This was followed by a media change and subsequent treatment with 10 μg/mL heparin or control, as well as 0.5 μL Golgi-Plug (GP) from BD biosciences. A CXCL10 ELISA from conditioned media was run after 6 hours of the initial treatment, and after 6 hours of the second treatment. In addition, the cell lysate was subjected to ELISA (FIG. 15). Again, heparin was shown to significantly increase the amount of CXCL10, as measured in the media, when compared to use of ADU-S100 alone, even when added subsequent to the ADU-S100.

### Example 16

It was also found that Heparin must be internalized to have an effect. B16F10 mouse melanoma cell lines were either treated with 50 μM ADU-S100+/−5 μg/mL heparin for 6 hours or with 50 μM ADU-S100+/−1 μg/mL heparin for 24 hours. Heparin-Sepharose beads (HEP-SEPH; Abcam) were also used per manufacturer's instructions at equivalent doses to unfractionated heparin. CXCL10 ELISAs from conditioned media were run (FIG. 16).

### Example 17

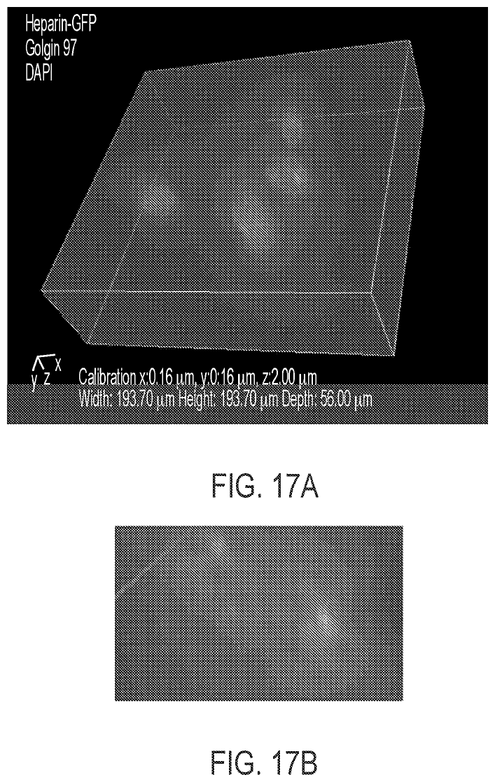

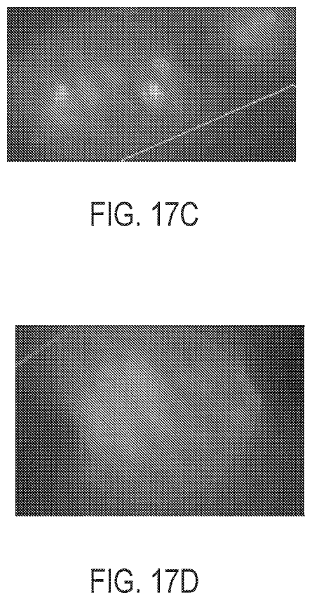

It was also found that Heparin does not co-localize with Golgi markers using immunofluorescence. MS428 human mesothelioma cells were grown in chamber slides (CelTreat) and treated for six hours with GFP-labeled heparin (Invitrogen) at 10 μg/ml. The samples were subjected to PFA fixing, methanol permeabilization, and staining with Golgin 97 antibody from Cell Signaling Technology (13192) per manufacturer's instructions at a dilution of 1:50 overnight. This was followed by goat anti-Rabbit IgG (H+L) Cross-Adsorbed Secondary Antibody, Alexa Fluor 555 (Invitrogen A21428) for 1 hour at 1:1000. Slides were mounted with anti-fade+DAPI and imaged using Z-stack on a Nikon Eclipse 80i microscope (FIGS. 17A-D). Co-localization was quantified from three high power fields and background from GFP-Heparin treated cells without Golgin antibody was subtracted before calculating the Pearson correlation co-efficient. The Blank-subtracted Pearson Correlation (r) was 0.14.

### Example 18

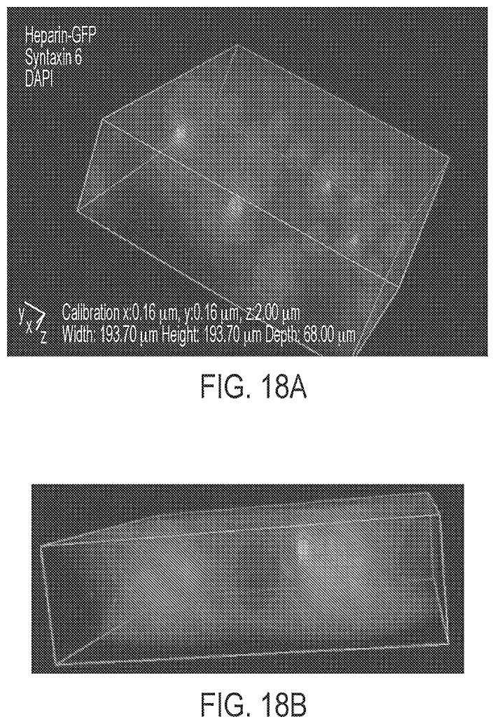

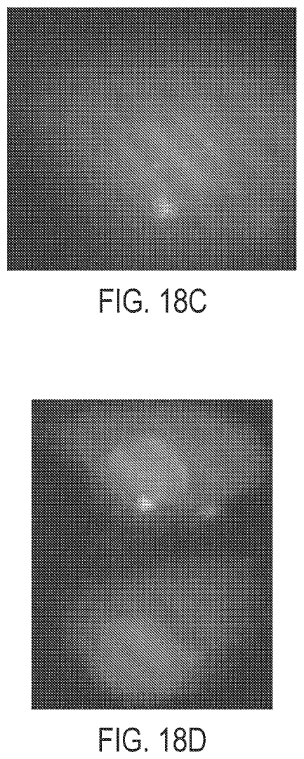

It was also found that Heparin co-localizes at some endosomes using immunofluorescence. MS428 human mesothelioma cells were grown in chamber slides (CelTreat) and treated for six hours with GFP-labeled heparin (Invitrogen) at 10 μg/ml. The samples were subjected to PFA fixing, methanol permeabilization, and staining with Syntaxin 6 antibody from Cell Signaling Technology (2869) per manufacturer's instructions at a dilution of 1:50 overnight. This was followed by goat anti-Rabbit IgG (H+L) Cross-Adsorbed Secondary Antibody, Alexa Fluor 555 (Invitrogen A21428) for 1 hour at 1:1000. Slides were mounted with anti-fade+DAPI and imaged using Z-stack on a Nikon Eclipse 80i microscope (FIGS. 18A-D). Co-localization was quantified from three high power fields and background from GFP-Heparin treated cells without Syntaxin antibody was subtracted before calculating the Pearson correlation co-efficient. The Blank-subtracted Pearson Correlation (r) was 0.32.

### Example 19

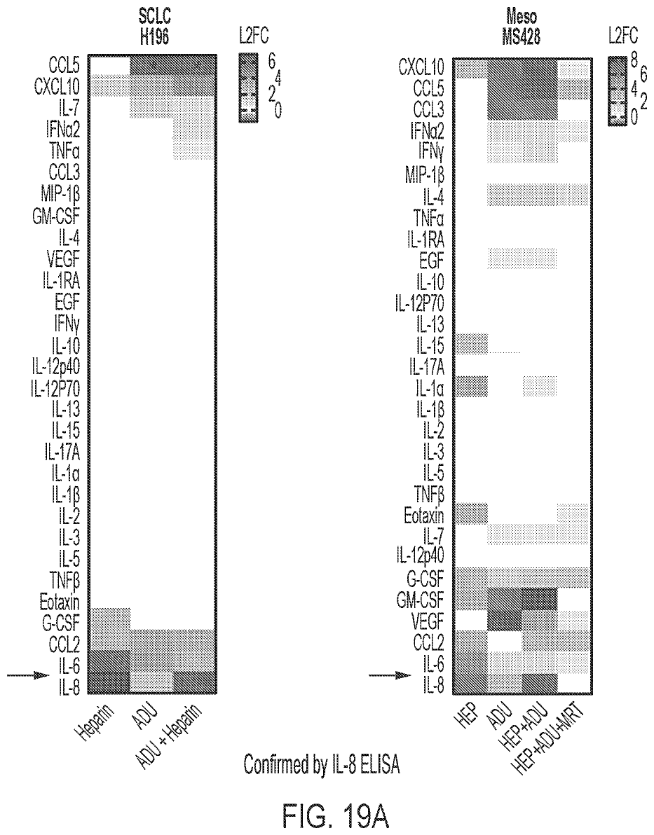

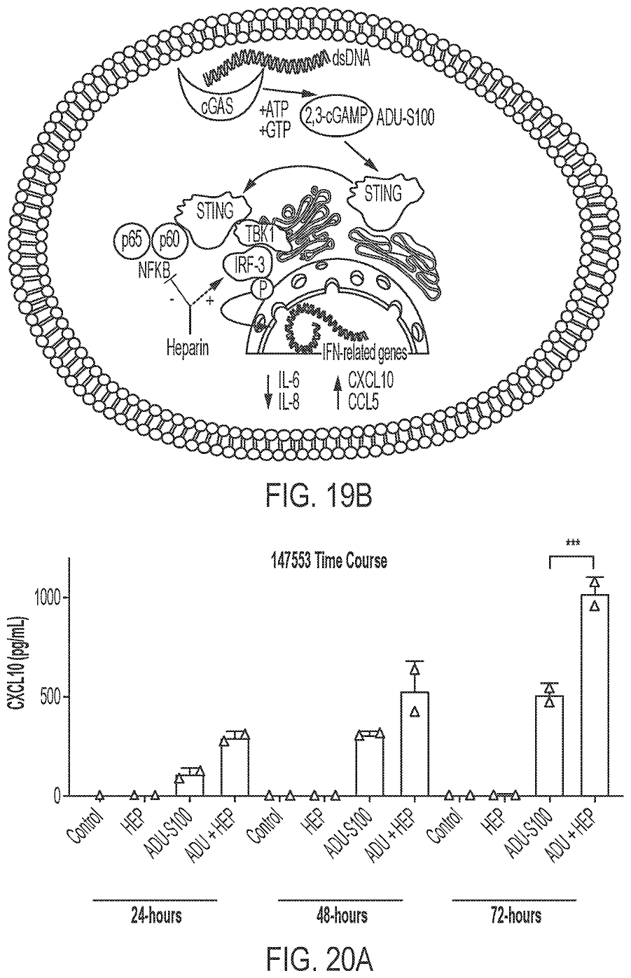

The influence of heparin (with and without a STING agonist) on IL-6 and IL-8 levels was examined. FIG. 19A shows the Luminex cytokine array after 24 hour treatment with 50 μM ADU+/−10 μg/mL heparin and 5 μM MRT TBK1 inhibitor in H196 SCLC and MS428 meningioma cells. The results showed an increase in T cell recruiting/growth suppressive cytokines such as CXCL10 and CCL5 and a decrease in growth-promoting cytokines such as IL-6 and IL-8 with the addition of heparin to ADU. This effect was reversed by MRT TBK1 inhibitor.

FIG. 19B shows a schematic illustrating that when administered alone a STING agonist upregulates NF-κB-associated cytokines (e.g., IL-6 and IL-8) and IFN related genes (e.g., CXCL10 and CCL5). However, as illustrated, the coadministration of heparin with STING agonist (e.g., ADU) increases phospho-IRF3 mediated upregulation of IFN related genes including the chemokines CXCL10 & CCL5 with concurrent decrease of NF-κB-associated cytokines IL-6 & IL-8 with the addition of heparin to STING agonists.

### Example 20

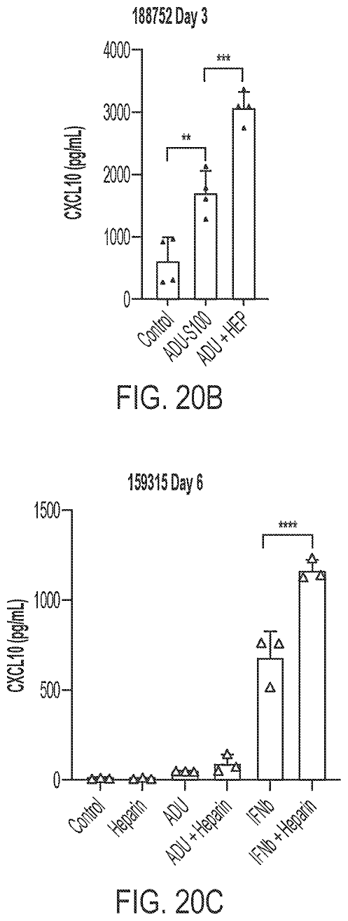

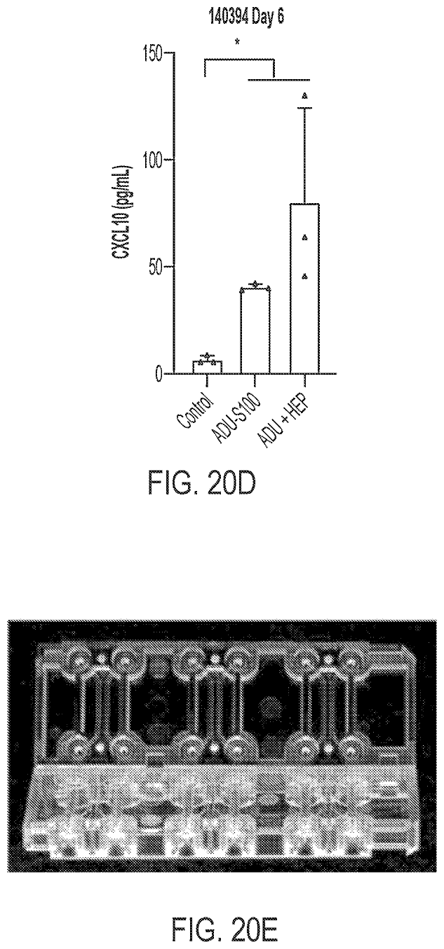

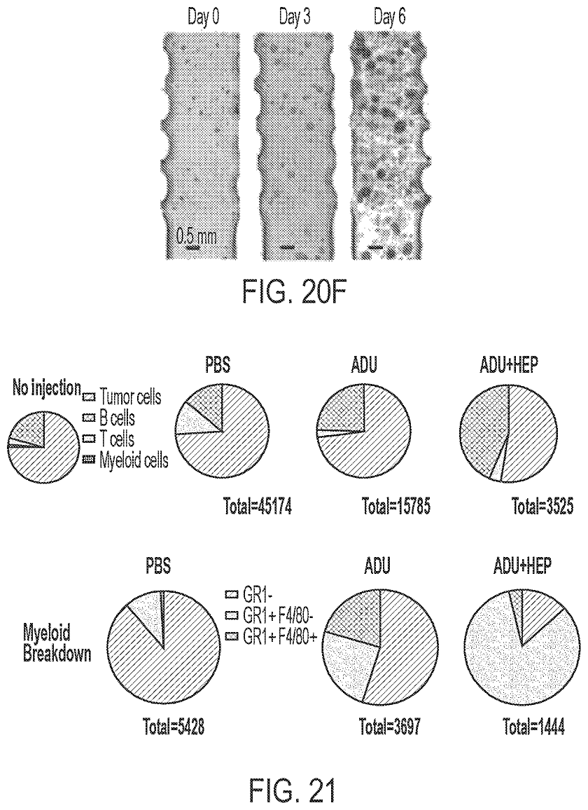

Patient-derived organotypic spheroids (PDOTs) were treated with 50 μM ADU-S100 +/−10 μg/mL heparin. A CXCL10 ELISA from conditioned media was run after 1-6 days of treatment (FIGS. 20A and 20E). Heparin was also shown to significantly increase the amount of CXCL10 in ex vivo cells, as measured in the media, when compared to use of ADU-S100 alone.

A similar result was seen when PDOTs were treated with 1 ng/ml IFNb+/−10 μg/mL heparin (FIG. 20F). A CXCL10 ELISA from conditioned media was run after 3 or 6 days of treatment. (FIG. 20B-20D). Heparin was also shown to significantly increase the amount of CXCL10 in ex vivo cells, as measured in the media, when compared to use of IFNb alone.

### Example 21

FIG. 21 shows Immune cell profiling from the 631 RPP mouse SCLC syngeneic model in BL6J. One tumor from each group was collected 3 days after intra-tumoral (IT) injection and processed using a Miltenyi dissociation kit prior to flow cytometry using a previously published panel of immune-cell antibodies.

### Materials and Methods for Examples

### Cell Culture and Treatments

H196, H69M, Lewis-Lung Carcinoma (LLC), H441, H1944, H2052, MS428, MS924, and MDA-MB-468 were cultured in RPMI (10% FBS, 1% penicillin). BEN-MEN-1, HBL52, GL261, CT2A, and B16F10 were cultured in DMEM (10% FBS, 1% penicillin). B16 Blue cells (Invivogen) were grown and used according to manufacturer's instructions. HUE and hLFBs were cultured in either complete Vasculife® or Fibrolife®, respectively. The cell culture media was changed as needed until confluence was reached, upon which the cells were split using 0.25% Trypsin-EDTA solution. For treatment, 1 mL of each cell line at a concentration of 300,000 cells/mL was plated in each well of a 12-well plate. The cell lines were then treated at varying doses of the clinical STING agonist ADU-S100 (ChemieTek), mammalian 2′,3′-cGAMP (InvivoGen), mouse and human interferons (R&D systems) and heparin (Sigma Aldrich), as specified in the figures. Desulfated heparins were purchased from Iduron, Fondaparinux and rivaroxaban from Selleck, and chondroitin sulfate from Sigma. Inhibitors used include MRT67307 and Ruxolitinib (Shanghai Haoyuan Chemexpress Co), Golgi Stop and Golgi Plug (BD Biosciences).

### Cytokine Profiling

Human and mouse CXCL10 ELISA (R&D Systems, SIP100, DY466) were performed according to the manufacturer's instructions. 1 mL of conditioned media from each treatment plate was collected after 24 hr culture unless otherwise specified. The collected media was centrifuged at 1400 RPM for 3 minutes to remove any cellular debris before use in the ELISA. Values represent the average of at least two independent biological replicates. For cell lysates, cells were collected on ice in Cell Lysis Buffer 2 (R&D systems), which is compatible with their ELISA kits.

Multiplex assays were performed utilizing the Human Cytokine/Chemokine Maganetic Bead Panel (Cat. #HCYTMAG-60K-PX30) on a Luminex MAGPIX system (Merck Millipore). Conditioned media concentrations (pg/ml) for each cytokine were derived from parameter curve fitting models. Fold changes relative to the corresponding control were calculated and plotted as log 2FC.

### Quantitative RT-PCR

RNA extraction was performed using the RNeasy Mini Kit (Qiagen, Cat. #74106). RNA samples (1000 ng) were reverse-transcribed into cDNA using Superscript® First-Strand Synthesis SuperMix (Thermo Fisher Scientific, Cat. #1683483). Quantitative real-time PCR was then performed using Power SYBR Green PCR Master Mix (Thermo Fisher Scientific, Cat. #4367659). The sequences of the primers used for qRT-PCR were obtained from previously published literature. Error bars represent technical replicates of each experiment.

### Patient-Derived Organotypic Spheroids

PDOTs were generated as described previously by Jenkins et al., Cancer Discovery 2018. Briefly, patient tumors collected through approved protocols were dissociated and loaded in collagen into microfluidic devices (AIM biotech). The side wells of each device were loaded with media containing the experimental treatments described in the figure legends. After 1-3 days, the media was collected and analyzed for cytokine levels using ELISA as described above.

### Small-Cell Lung Cancer Syngeneic Mouse Model and Flow Cytometry

631 RPP (RPP) SCLC mouse cell lines were derived from SCLC tumors that were generated in LSL-Cas9 BL6 mice that were intratracheally injected with AAV that encode Cre-recombinase and sgRNAs targeting Rb1, Trp53, and Rb12 (RPP) as described in Oser et al., Genes Dev, 2019. These cells were re-implanted in the flank of BL/6 mice and allowed to form tumors of approximately 300 mm3 before intra-tumoral injection with 50 μg ADU-S100 +/−10 μg heparin. After 72 hours, mice were euthanized with CO2, their tumors quickly extracted and dissociated using a Miltenyi kit prior to flow cytometry with a panel of antibodies against mouse immune cells as previously described in Jenkins et al., Cancer Discovery, 2018.

### Statistical Analysis

GraphPad Prism 8.0 was used for statistical analysis, data processing, and graph generation. Values reported are the mean and SEM. When comparing only two groups, a Student t test was applied; otherwise, an ANOVA multivariate analysis was performed with a post hoc modification as described in the figure legends. Statistical significance was determined as P<0.05.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. A method of treating a subject having cancer, comprising:
administering to the subject a therapeutically effective amount of a stimulator of interferon signaling and a therapeutically effective amount of a heparin polysaccharide, wherein the heparin polysaccharide has reduced anticoagulant activity.

2. The method of claim 1, wherein the heparin polysaccharide is at least one of desulfated and N-acetylated.

3. The method of claim 2, wherein the heparin polysaccharide is at least one of N-desulfated and O-desulfated.

4. The method of claim 2 or 3, wherein the heparin polysaccharide is at least one of 2-O desulfated, 3-O desulfated, and 6-O desulfated.

5. The method of any one of claims 1-4, wherein the heparin polysaccharide comprises a glycol-split monomer.

6. The method of any one of claims 1-5, wherein the heparin polysaccharide lacks a unique pentasaccharide sequence, wherein the unique pentasaccharide sequence has the following general structure:

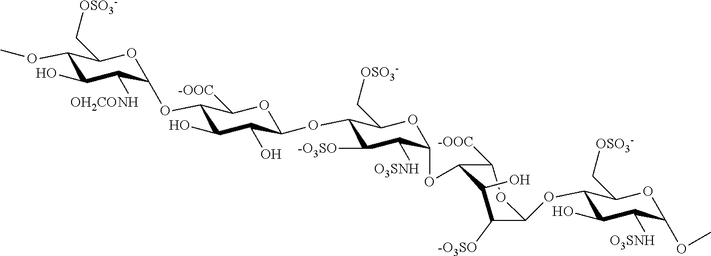

7. The method of any one of claims 1-6, wherein the heparin polysaccharide is administered locally, intratumorally, or systemically.

8. The method of any one of claims 1-7, wherein the stimulator of interferon signaling agonist is administered locally, intratumorally, or systemically.

9. The method of any one of claims 1-8, wherein the heparin polysaccharide is low molecular weight heparin.

10. The method of any one of claims 1-9, wherein the stimulator of interferon signaling is selected from the group consisting of interferon alpha, interferon beta, STING agonists, TLR agonists, and oncolytic viruses.

11. The method of claim 10, wherein the STING agonists is selected from the group consisting of cyclic GMP-AMP (cGAMP), ganciclovir, ADU-S100, and CMA.

12. The method of any one of claims 1-11, further comprising:
administering to the subject a chemotherapeutic agent.

13. The method of claim 12, wherein the chemotherapeutic agent is a checkpoint inhibitor.

14. The method of claim 12 or 13, wherein the chemotherapeutic agent is a programed cell death protein 1 (PD-1) inhibitor or a programed death-ligand 1 (PD-L1) inhibitor.

15. The method of any one of claims 1-14, wherein the cancer is selected from the group consisting of carcinoma, lymphoma, blastoma, sarcoma, and leukemia.

16. The method of any one of claims 1-15, wherein the cancer is selected from the group consisting of cancers of the lung, bone, pancreas, skin, head, neck, uterus, ovaries, stomach, colon, breast, esophagus, small intestine, bowel, endocrine system, thyroid gland, parathyroid gland, adrenal gland, urethra, prostate, penis, testes, ureter, bladder, kidney or liver; rectal cancer, cancer of the anal region, carcinomas of the fallopian tubes, endometrium, cervix, vagina, vulva, renal pelvis, renal cell, sarcoma of soft tissue, myxoma, rhabdomyoma, fibroma, lipoma, teratoma, cholangiocarcinoma, hepatoblastoma, angiosarcoma, hemangioma, hepatoma, fibrosarcoma, chondrosarcoma, myeloma, chronic or acute leukemia, lymphocytic lymphomas, primary CNS lymphoma, neoplasms of the CNS, spinal axis tumors, squamous cell carcinomas, synovial sarcoma, malignant pleural mesotheliomas, brain stem glioma, pituitary adenoma, meningioma, bronchial adenoma, chondromatous hanlartoma, inesothelioma, Hodgkin's Disease, brain (gliomas), glioblastomas, astrocytomas, glioblastoma multiforme, Bannayan-Zonana syndrome, Cowden disease, Lhermitte-Duclos disease, Wilm's tumor, Ewing's sarcoma, Rhabdomyosarcoma, ependymoma, medulloblastoma, melanoma, ovarian, pancreatic, adenocarcinoma, ductal madenocarcinoma, adenosquamous carcinoma, small cell lung cancer, acinar cell carcinoma, glucagonoma, insulinoma, prostate, sarcoma, osteosarcoma, giant cell tumor of bone, thyroid, lymphoblastic T cell leukemia, chronic myelogenous leukemia, chronic lymphocytic leukemia, hairy-cell leukemia, acute lymphoblastic leukemia, acute myelogenous leukemia, chronic neutrophilic leukemia, acute lymphoblastic T cell leukemia, plasmacytoma, Immunoblastic large cell leukemia, mantle cell leukemia, multiple myeloma, megakaryoblastic leukemia, multiple myeloma, acute megakaryocyte leukemia, pro myelocytic leukemia, erythroleukemia, malignant lymphoma, hodgkins lymphoma, non-hodgkins lymphoma, lymphoblastic T cell lymphoma, Burkitt's lymphoma, follicular lymphoma, neuroblastoma, bladder cancer, urothelial cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor) and testicular cancer.

17. The method of any one of claims 1-14, wherein the cancer is selected from the group consisting of small cell lung cancer, non-small cell lung cancer, mesothelioma, meningioma, and triple negative breast cancer.

18. A method of treating a subject having cancer, comprising:
administering to the subject a therapeutically effective amount of a stimulator of interferon signaling and a therapeutically effective amount of a heparin polysaccharide, wherein the subject is not receiving concurrent antithrombotic therapy or thrombolytic therapy.

19. The method of claim 18, wherein the heparin polysaccharide is at least one of desulfated and N-acetylated.

20. The method of claim 18 or 19, wherein the heparin polysaccharide is low molecular weight heparin.

21. The method of any one of claims 18-20, wherein the antithrombotic therapy is an anticoagulant therapy.

22. The method of any one of claims 18-21, wherein the cancer is meningioma, glioma, medulloblastoma, pituitary adenomas, primary CNS lymphomas, or a cancer associated with CNS germ cell tumors.

23. The method of any one of claims 18-22, wherein the cancer is small cell lung cancer or a non-small cell lung cancer.

24. The method of any one of claims 18-23, wherein the subject is undergoing surgery on the brain or central nervous system (CNS).

25. The method of any one of claims 18-24, wherein the subject has or is at risk of having intracranial bleeding, hepatic damage or hepatic failure.

26. The method of any one of claims 18-25, wherein the heparin polysaccharide is administered locally, intratumorally, or systemically.

27. The method of any one of claims 18-26, wherein the stimulator of interferon signaling is administered locally, intratumorally, or systemically.

28. The method of any one of claims 18-27, wherein the stimulator of interferon signaling is selected from the group consisting of interferon alpha, interferon beta, STING agonists, TLR agonists, and oncolytic viruses.

29. The method of claim 28, wherein the STING agonist is selected from the group consisting of cyclic GMP-AMP (cGAMP), ganciclovir, ADU-S100, and CMA.

30. The method of any one of claims 18-29, further comprising:
administering to the subject a chemotherapeutic agent.

31. The method of any one of claim 30, wherein the chemotherapeutic agent is a checkpoint inhibitor.

32. The method of claim 30 or 31, wherein the chemotherapeutic agent is a programed cell death protein 1 (PD-1) inhibitor or a programed death-ligand 1 (PD-L1) inhibitor.

33. A method of treating a subject having cancer, comprising:
administering to the subject a therapeutically effective amount of stimulator of interferon signaling and a therapeutically effective amount of a heparin polysaccharide, wherein the heparin is administered locally to the cancer or intratumorally.

34. The method of claim 33, wherein the stimulator of interferon signaling is administered locally to the cancer, intratumorally, or systemically.

35. The method of claim 33 or 34, wherein the stimulator of interferon signaling is selected from the group consisting of interferon alpha, interferon beta, STING agonists, TLR agonists, and oncolytic viruses.

36. The method of claim 35, wherein the STING agonist is selected from the group consisting of cyclic GMP-AMP (cGAMP), ganciclovir, ADU-S100, and CMA.

37. The method of any one of claims 33-36, further comprising:
administering to the subject a chemotherapeutic agent.

38. The method of claim 37, wherein the chemotherapeutic agent is a checkpoint inhibitor.

39. The method of claim 37 or 38, wherein the chemotherapeutic agent is a programed cell death protein 1 (PD-1) inhibitor or a programed death-ligand 1 (PD-L1) inhibitor.

40. The method of any one of claims 33-39, wherein the cancer is selected from the group consisting of carcinoma, lymphoma, blastoma, sarcoma, and leukemia.

41. The method of any one of claims 33-40, wherein the cancer is selected from the group consisting of cancers of the lung, bone, pancreas, skin, head, neck, uterus, ovaries, stomach, colon, breast, esophagus, small intestine, bowel, endocrine system, thyroid gland, parathyroid gland, adrenal gland, urethra, prostate, penis, testes, ureter, bladder, kidney or liver; rectal cancer, cancer of the anal region, carcinomas of the fallopian tubes, endometrium, cervix, vagina, vulva, renal pelvis, renal cell, sarcoma of soft tissue, myxoma, rhabdomyoma, fibroma, lipoma, teratoma, cholangiocarcinoma, hepatoblastoma, angiosarcoma, hemangioma, hepatoma, fibrosarcoma, chondrosarcoma, myeloma, chronic or acute leukemia, lymphocytic lymphomas, primary CNS lymphoma, neoplasms of the CNS, spinal axis tumors, squamous cell carcinomas, synovial sarcoma, malignant pleural mesotheliomas, brain stem glioma, pituitary adenoma, meningioma, bronchial adenoma, chondromatous hanlartoma, inesothelioma, Hodgkin's Disease, brain (gliomas), glioblastomas, astrocytomas, glioblastoma multiforme, Bannayan-Zonana syndrome, Cowden disease, Lhermitte-Duclos disease, Wilm's tumor, Ewing's sarcoma, Rhabdomyosarcoma, ependymoma, medulloblastoma, melanoma, ovarian, pancreatic, adenocarcinoma, ductal madenocarcinoma, adenosquamous carcinoma, small cell lung cancer, acinar cell carcinoma, glucagonoma, insulinoma, prostate, sarcoma, osteosarcoma, giant cell tumor of bone, thyroid, lymphoblastic T cell leukemia, chronic myelogenous leukemia, chronic lymphocytic leukemia, hairy-cell leukemia, acute lymphoblastic leukemia, acute myelogenous leukemia, chronic neutrophilic leukemia, acute lymphoblastic T cell leukemia, plasmacytoma, Immunoblastic large cell leukemia, mantle cell leukemia, multiple myeloma, megakaryoblastic leukemia, multiple myeloma, acute megakaryocyte leukemia, pro myelocytic leukemia, erythroleukemia, malignant lymphoma, hodgkins lymphoma, non-hodgkins lymphoma, lymphoblastic T cell lymphoma, Burkitt's lymphoma, follicular lymphoma, neuroblastoma, bladder cancer, urothelial cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor) and testicular cancer.

42. The method of any one of claims 33-39, wherein the cancer is selected from the group consisting of small cell lung cancer, non-small cell lung cancer, mesothelioma, meningioma, and triple negative breast cancer.

43. A pharmaceutical composition for the treatment of cancer, comprising a stimulator of interferon signaling, a heparin polysaccharide, and a pharmaceutically acceptable excipient.

44. The pharmaceutical composition of claim 43, wherein the heparin polysaccharide has reduced anticoagulant activity.

45. The pharmaceutical composition of claim 43 or 44, wherein the heparin polysaccharide is at least one of desulfated and N-acetylated.

46. The pharmaceutical composition of claim 45, wherein the heparin polysaccharide is at least one of N-desulfated and O-desulfated.

47. The pharmaceutical composition of claim 45 or 46, wherein the heparin polysaccharide is at least one of 2-O desulfated, 3-O desulfated, and 6-O desulfated.

48. The pharmaceutical composition of any one of claims 43-47, wherein the heparin polysaccharide comprises a glycol-split monomer.

49. The pharmaceutical composition of any one of claims 43-48, wherein the heparin polysaccharide is low molecular weight heparin.

50. The pharmaceutical composition of any one of claims 43-49, wherein the heparin polysaccharide lacks a unique pentasaccharide sequence, wherein the unique pentasaccharide sequence has the following general structure:

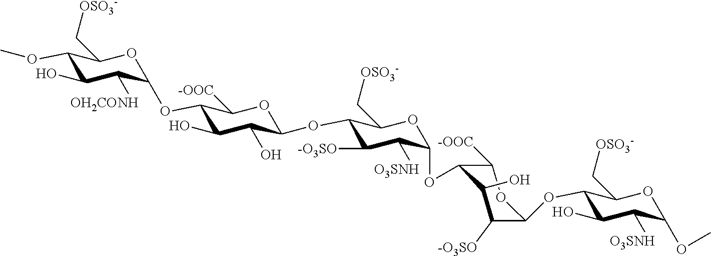

51. The pharmaceutical composition of any one of claims 43-50, wherein the stimulator of interferon signaling is selected from the group consisting of interferon alpha, interferon beta, STING agonists, TLR agonists, and oncolytic viruses.

52. The method of claim 51, wherein the STING agonist is selected from the group consisting of cyclic GMP-AMP (cGAMP), ganciclovir, ADU-S100, and CMA.

53. The pharmaceutical composition of any one of claims 43-52, wherein the pharmaceutically acceptable excipient is water or saline.

54. The method or pharmaceutical composition of any one of the preceding claims, wherein the heparin polysaccharide does not comprise a synthetic pentasaccharide.

55. The method of pharmaceutical composition of any one of the preceding claims, wherein the heparin polysaccharide does not comprise fondaparinux.

56. A method of treating a subject having cancer, comprising:
administering to the subject a therapeutically effective amount of an innate immunity therapy and a therapeutically effective amount of a heparin polysaccharide, wherein the heparin polysaccharide has reduced anticoagulant activity.

57. The method of claim 56, wherein the innate immunity therapy comprises an agent that stimulates CD8 T cell activation.

58. The method of claim 57, wherein the agent that stimulates CD8 T cell activation is a 4-1BB agonist.

59. The method of claim 57, wherein the agent that stimulates CD8 T cell activation is an OX40 agonist.

60. The method of claim 56, wherein the innate immunity therapy comprises a tumor vaccine.

61. The method of claim 56, wherein the innate immunity therapy comprises adoptive cell transfer.
